(19) United States
(12) Patent Application Publication
(10) Pub. No.: US 2025/0011716 A1
(43) Pub. Date: Jan. 9, 2025

(54) KNITTED CELL SCAFFOLDS

(71) Applicant: Massachusetts Institute of Technology, Cambridge, MA (US)

(72) Inventors: Steven R. Gillmer, Somerville, MA (US); Ming Guo, Lexington, MA (US); Erin Doran, Waltham, MA (US); Emily Holtzman, Cambridge, MA (US); Ariel Sandberg, Arlington, MA (US); Matthew Bernasconi, Maynard, MA (US)

(73) Assignee: Massachusetts Institute of Technology, Cambridge, MA (US)

(21) Appl. No.: 18/393,618

(22) Filed: Dec. 21, 2023

Related U.S. Application Data
(60) Provisional application No. 63/434,896, filed on Dec. 22, 2022.

(51) Int. Cl. C12N 5/073; A61L 27/16; A61L 27/24; A61L 27/38; A61L 27/56; A61L 27/58

(52) U.S. Cl. CPC C12N 5/0603; A61L 27/16; A61L 27/24; A61L 27/3813; A61L 27/3817; A61L 27/3821; A61L 27/3826; A61L 27/3834; A61L 27/3839; A61L 27/3847; A61L 27/3852; A61L 27/386; A61L 27/3873; A61L 27/56; A61L 27/58; A61L 2430/02; A61L 2430/06; A61L 2430/10; A61L 2430/30; A61L 2430/34; C12N 2533/30; C12N 2533/54; C12N 2537/00

(57) ABSTRACT

Knit scaffolds for culturing cells and aiding in the healing of functional tissue are provided. The knit scaffolds have properties aligned with specific tissues for providing optimal tissue growing surfaces including matched biomechanical properties. The scaffolds are made up of knitted material and do not require or utilize a support skeleton for function. Methods of making and using the knit scaffolds are also provided.

### RELATED APPLICATIONS

This application claims the benefit under 35 U.S.C. § 119(e) of U.S. provisional application No. 63/434,896, filed Dec. 22, 2022, the entire contents of which are incorporated herein by reference.

### GOVERNMENT FUNDING

This invention was made with government support under FA8702-15-D-0001 awarded by the U.S. Air Force. The government has certain rights in the invention.

### BACKGROUND

Expedited recovery of soft tissues can be mediated by seeding stem cells or other relevant extracted cells onto biocompatible scaffolds. These capabilities can be exceptionally valuable not only for civilian applications, but military as well. Current state-of-the-art scaffolds have been used to support cell therapy for soft tissue recovery. These scaffolds commonly are made from collagen, fibrin, or other natural or artificial hydrogel-based substrates which host living cells. An ideal scaffold mimics native tissues like skin and muscles, which comprise stromal cells and protein scaffolds, and can maintain cell functions even under a severe strain.

A common problem in the development of state-of-the-art scaffolds is that the seeded cells detach and/or die when the supporting scaffold strains, leading to unexpected immune response, inflammation, and secondary injury during rehabilitation. The adverse side effects can be a result of a variety of cell death mechanisms including nuclear envelope rupture, apoptosis, and DNA damage. A solution to this problem is to implement a stiff scaffold to avoid cell stretching and the resulting cell death. Inherently, however, a stiff scaffold is impractical because it cannot follow the natural motions of the subject it aims to heal. An overly stiff scaffold combined with naturally flexible tissue creates a mismatch in biomechanical elasticity adding increased stress of the cells during recovery. Conversely, overly-compliant scaffolds do not sufficiently support injured tissue during the healing phase. To maximize healing efficiency, scaffolds seeded with cells should be matched to biomechanical properties of the injured tissue they are aiming to heal.

### SUMMARY

In some aspects, provided herein is a three-dimensional knit scaffold (also referred to herein as “knit scaffold”) comprising biocompatible fibers arranged in a knit sequence and comprising a microscopic and macroscopic porosity, wherein the scaffold has elasticity and anisotropy values matched to a target tissue.

In some embodiments, the scaffold has three characteristic regions of a stress/strain curve which mimic natural tissue and correspond to an uncrimping of the knit, transitioning to stretch, and stretching of the fibers which compose the knit.

In some embodiments, the target tissue is skin comprising a matched knit scaffold elasticity value of between 5 Pa to 10 MPa, and a skin anisotropy value is between 1:1.01 to 1:10. In some embodiments, the target tissue is ligament comprising a matched knit scaffold elasticity value of between 100 kPa to 10 GPa, and a ligament anisotropy value is between 1:1.01 to 1:10. In some embodiments, the target tissue is muscle comprising a matched knit scaffold elasticity value of between 1 kPa to 10 MPa, and a muscle anisotropy value is between 1:1.01 to 1:10. In some embodiments, the target tissue is cartilage comprising a matched knit scaffold elasticity value of between 1 kPa to 10 MPa, and a cartilage anisotropy value is between 1:1.01 to 1:10. In some embodiments, the target tissue is adipose comprising a matched knit scaffold elasticity value of between 1 Pa to 100 kPa, and an adipose anisotropy value is between 1:1.01 to 1:10. In some embodiments, the target tissue is bone comprising a matched knit scaffold elasticity value of between 10 MPa to 200 GPa, and a bone anisotropy value is between 1:1.01 to 1:10. In some embodiments, a particular elasticity value of the knit scaffold occurs at any point of the three characteristic regions of the stress/strain curve.

In some embodiments, cells grow on the knit scaffold. In some embodiments, the three-dimensional knit scaffold further comprises cells growing on the scaffold. In some embodiments, the three-dimensional knit scaffold has Type I, Type II, Type III, Type IV, and/or Type V collagen added to the surface of the scaffold before cells are seeded on the scaffold. In some embodiments, fibrin is added to the knit scaffold before cells are seeded on the scaffold. In some embodiments, hydrogel is added to the knit scaffold before cells are seeded on the scaffold.

In some embodiments, a knit structure, knit type, stitch length, number of filaments in a single ‘end’ of yarn, number of ends plied together in a single yarn, filament denier, filament diameter, filament cross sectional shape, yarn texture, overall yarn size, and/or material vary to match the biomechanical properties of the target tissue. In some embodiments, the yarn is made with continuous filaments or twisted fiber staples. In some embodiments, the knit type is selected from the group consisting of jersey knit, ribbed knit, and interlock knit. In some embodiments, the knit type is a combination of different knit types, purl, miss or float, tuck, and the transfer of stitches. In some embodiments, the biocompatible fiber is selected from the group comprising polylactic acid (PLA), polyglycolic acid (PGA), polylactic-co-glycolic acid (PLGA), poly-l-lactic acid (PLLA), poly-d-lactic acid (PLDA), Polydioxanone (PDS or PDO), Polycaprolactone (PCL), Trimethylene carbonate (TMC), or blends thereof. In some embodiments, the scaffold is comprised of a combination of PLLA, PLDA, and/or PLGA with L-Lactide (PLLA) and D-lactide (PLDA) crystallinities both ranging from 0% to 100% crystallinity. In some embodiments, the scaffold is comprised of silk and may or may not be blended with selections from the group comprising polylactic acid (PLA), polyglycolic acid (PGA), polylactic-co-glycolic acid (PLGA), poly-l-lactic acid (PLLA), poly-d-lactic acid (PLDA), Polydioxanone (PDS or PDO), Polycaprolactone (PCL), Trimethylene carbonate (TMC). In some embodiments, the scaffold has a stitch length of 1 mm to 25 mm. In some embodiments, the scaffold has a number of filaments combined into a single ‘end’ of fiber ranging from 1-500 or 1-300 filaments. In some embodiments, the scaffold has a number of fiber ends plied together in a single yarn having a value of 1-10. In some embodiments, the scaffold has yarns that are flat, air textured, false twisted, knit-de-knit, or edge crimped. In some embodiments, the scaffold has filaments with diameters of 0.001 mm to 4 mm or 0.1 mm to 2 mm. In some embodiments the scaffold is comprised of yarns with sizes of 5-1000 denier. In some embodiments, the scaffold has filaments with a cross section shape of circular, hollow, or non-circular. In some embodiments, the scaffold has yarns consisting of single component, bi-component or multi-component filaments. In some embodiments, the scaffold has a degradation rate of 1 week to 3, 4 or 5 years.

In some aspects, provided herein is a method for preparing a tissue scaffold, comprising identifying a set of values defining an elasticity, ultimate strength, anisotropy, healing rate, and/or hydraulic permeability value of a target tissue, and knitting mono-filament or multi-filament biocompatible fibers to produce a scaffold arranged in a knit sequence and comprising a microscopic porosity sufficient to nurture growing cells compatible with target tissue recovery, macroscopic porosity matched to the required hydraulic permeability of the target tissue, degradation rate which degrades at a similar rate to the healing rate of the target tissue, and having anisotropy, ultimate strength, and biomechanical elasticity values matched to that of the target tissue.

In some aspects, the shape and/or pattern of a knit scaffold matches the area of the body where the scaffold is implemented. Taken as an example, in some embodiments, the shape and/or pattern of a knit scaffold wherein the target tissue is the hand is shaped like a glove. In some embodiments, the shape and/or pattern of a knit scaffold wherein the target tissue is a foot is shaped like a shoe. The shape and/or pattern of the knit scaffold may be any shape suitable for compatibility with the target tissue.

In some embodiments, the method further comprises seeding the scaffold with human-derived tissue, blood, bone marrow, platelet rich plasma, platelets, mononuclear cells, progenitor cells, inflammatory cells, primary cells, stem cells, induced pluripotent stem cells or purified cell populations, fluids, or proteins. In some embodiments, the method further comprises seeding the scaffold with cells selected from the group consisting of chondrocytes, osteoblasts, fibroblasts, angioblasts, myoblasts, epithelial cells, urothelial cells, smooth muscle cells, keratinocytes, beta cells, endothelial cells, fibrocytes, vascular endothelial cells, hepatocytes, small intestine epithelial cells, epidermal keratinocytes, bone marrow mesenchymal cells, cardiomyocytes, intervertebral disc cells, oral mucosal epithelia, gastrointestinal mucosal epithelia, urinary tract epithelia, skeletal joint synovium, periosteum, perichondrium, skeletal muscle cells, smooth muscle cells, cardiac muscle cells, pericardium, dura, meninges, keratinocyte precursor cells, pericytes, glial cells, neural cells, amniotic membrane, placental membrane, serosal cells, undifferentiated stem cells, undifferentiated progenitor cells, pre-differentiated stem cells, pre-differentiated progenitor cells, neural stem cells, neural progenitor cells, neuronal cells, dendritic cells, genetically engineered cells, and/or stem cells. In some embodiments, the stem cells are selected from the group consisting of hematopoietic, mesenchymal, postpartum, pancreatic, hepatic, retinal epithelial, olfactory bulb, endothelial, muscle, adipose derived, ileac crest, bone marrow, periodontal ligament, oval and dermal cells and organ specific stem and/or progenitor cells.

In some aspects, disclosed herein is a method of treating a subject in need of tissue generation comprising contacting the subject with a scaffold and allowing tissue to generate on the scaffold. In some embodiments, the subject is a human. In some embodiments, the tissue is skin, muscle, ligament, cartilage, adipose, and/or bone.

### BRIEF DESCRIPTIONS OF THE DRAWINGS

Non-limiting embodiments will be described by way of example with reference to the accompanying figures, which are schematic and are not intended to be drawn to scale. For purposes of clarity, not every component is labeled in every figure, nor is every component of each embodiment of the invention shown where illustration is not necessary to allow those of ordinary skill in the art to understand the invention.

FIGS. 1A-1B provide an overview of how, when strained, the uncrimping of a knitted cell scaffold is advantageous over the stretching of a cell scaffold with no initial uncrimping region, e.g. scaffolds comprised of a collagen, fibrin, or other hydrogel-based growth layer do not typically uncrimp initially. FIG. 1A shows a qualitative representation of the stress levels that cells experience in native tissue when the naturally occurring structural filaments (i.e., collagen fibers) undergo strain. The nonlinearity in the curve is due to the ability of collagen fibers and the elastin matrix within tissue to uncrimp instead of stretch immediately after strain begins. FIG. 1B shows a qualitative comparison of the stretch experienced by cells that are seeded on a cell scaffold comprised of a traditional-based growth layer (top panel) versus a knitted cell scaffold (bottom panel). The knit scaffold minimizes local strain on seeded cells through an uncrimping of the underlying yarn scaffold, similar to native tissue. This effect thereby prevents the cells from undergoing harmful levels of deformations.

FIG. 2 provides an overview of different knit types and parameters that can be varied herein, and demonstrates the conformation of the underlying yarn when using interlock, ribbed, or jersey-type knits. FIG. 2 also depicts the directionality that is referred to when referencing the “wales” and “courses” of the knits. The stitch length, also sometimes called loop length or yarn consumed per stitch, can be varied according to a knitting machine's stich cam setting to adjust properties of the knits. For Stoll flat knitting machines, the stitch cam setting is represented by the Needle Placement (NP) value. The higher is the NP value, the larger loop length is knitted. The NP value is referenced in subsequent figures. Not shown on this Figure, but subject to the claims herein, is how the elastic properties of the knits can be adjusted by varying the number of filaments integrated in to the base yarn in addition to altering the material of the filaments themselves.

FIGS. 3A-3D demonstrate the ability to culture, grow and image live cells on the knitted cell scaffold device described herein. FIG. 3A shows a high-magnification image of an interlock knit scaffold comprised of PLA. FIG. 3B shows a high-magnification image of live cells growing on an interlock knit scaffold. FIG. 3C shows a high-magnification image of live cells growing on an interlock knit scaffold, wherein the scaffold itself was not included in the imaging. FIG. 3D shows images of the experimental setup used to capture the images shown in FIGS. 3A-C.

FIG. 4 shows a graph reporting the survival capabilities of cells seeded on knit scaffolds with varying knit types at a bulk strain amplitude of 30%. Mouse embryonic fibroblasts were seeded on the knit scaffolds and were then subjected to bulk strain amplitudes of 30% cyclically at approximately 1 Hz for 10 minutes. The live cells were imaged and counted before straining to create a control. After cyclic strain, the cells were again imaged and re-counted to calculate a survival percentage relative to the initial conditions. All knits in these results used PLA multifilament yarn and controlled stitch length with a Needle Placement (NP) value of 13 on a Stoll flat knitting machine.

FIG. 5 shows a graph reporting the survival capabilities of cells seeded on interlock knit scaffolds, plotted as a function of variable stitch lengths, as controlled by the stitch cam setting (NP value on this particular unit) of the machine, stressed in the courses or wales directions. The seeded cells were mouse stem cells and were stretched for approximately 10 minutes at 1 Hz, 50% strain. The plotted percentages indicate the number of live cells, post-stretching.

FIGS. 6A-6C show the results of an extended loading/unloading test. Two interlock knits with 11 NP stitch length (as controlled by the stitch cam setting of the knitting machine) were stretched in orthogonal wales and courses directions at set strain levels (FIG. 6A). The initial over-stretch to 60% demonstrates that subsequent tests exhibit lower stress responses with repeatable load/unload curves (FIGS. 6B-6C). The knits used were PLA Interlock with 11 NP as the stitch cam setting from the knitting machine.

FIGS. 7A-7B show graphs plotting stress of varying types of knitted filaments (jersey, interlock, or ribbed) against a range of strain percentages. The stitch length was kept nominally constant throughout all tests by fabricating samples, extracting fibers, and measuring physical stitch lengths. The stitch cam settings of 10 NP for interlock, 10 NP for ribbed, and 13 NP for jersey produced stitch lengths of 5.97 mm, 6.02 mm, and 6.11 mm, respectively (stitch length defined as yarn length per stitch). The strain was introduced in either the wales (FIG. 7A) or courses (FIG. 7B) directions with 3 samples per knit type (9 samples total per direction). The strain was continuously applied and the scaffolds stretched until failure while measuring the reaction force with an Instron® machine. Changes to knit type primarily change the stiffness of the knits in the wales direction with Ribbed as most compliant, followed by Interlock and Jersey exhibiting progressively stiffer responses. Changes to knit type therefore adjust elasticity and anisotropy of knits to match a target tissue's properties.

FIGS. 8A-8B show graphs plotting stress of variable-stitch length interlock knits against a range of strain percentages. The knit type was held as Interlock with variable NP values as controlled by the stitch cam setting. The strain was introduced in either the wales (FIG. 8A) or courses (FIG. 8B) directions with 3 samples per knit type (9 samples total per direction). The strain was continuously applied and the scaffolds stretched until failure while measuring the reaction force with an Instron® machine. Changes to stitch length primarily change the stiffness of the knits in the courses direction with 11 NP as most compliant, followed by 10 NP and 9 NP exhibiting progressively more stiff responses. Changes to stitch length therefore adjust elasticity and anisotropy of knits to match a target tissue's properties.

FIGS. 9A-9B show graphs plotting stress of variable numbers of “ends” against a range of strain percentages. Each “end” of yarn is comprised of is 64 filaments of 167 dtex PLA yarn. The knit type was held as Interlock with variable ends plied together before knitting. The strain was introduced in either the wales (FIG. 9A) or courses (FIG. 9B) directions with 3 samples per knit type (9 samples total per direction). The strain was continuously applied and the scaffolds stretched until failure while measuring the reaction force with an Instron® machine. Changes to number of ends primarily change the ultimate strength of the knits in the courses and wales directions with 4 ends exhibiting the highest strength, followed by 3 ends and 2 ends exhibiting progressively lower ultimate strength. Changes to knit type therefore be used to change the mechanical properties of knits to match a target tissue's properties.

FIGS. 10A-10B show graphs depicting the average porosity of interlock knits as a function of stitch length (controlled in NP, the needle placement value as controlled by the stitch cam setting; FIG. 10A) and ends plied together (each “end” of yarn is comprised of is 64 filaments of 167 dtex PLA yarn; FIG. 10B). Porosity is defined as the ratio (presented as a percentage) of the porous media divided by the same volume of a full uniform material. Thickness of the materials were measured per ASTM D1777. Changes to stitch length and number of ends plied together can therefore be used to control the porosity of the knits.

### DETAILED DESCRIPTION

Many native tissues such as skin, muscle, tendon and ligament exhibit exceptional mechanical performance, including high stretchability and toughness, to withstand substantial internal and external mechanical loads. Fabrication of tissue constructs with comparable mechanical properties to native tissues is desired for applications in translational medicine and tissue engineering. Toward this end, a variety of synthetic biomaterials have been developed to achieve high stretchability and toughness. For example, double-network elastomers with both ionic and covalent bonds can be stretched beyond 20 times their initial length. Nano-composite hydrogels can also be highly stretchable and have a fracture energy of ˜10.1 MJ m⁻². The merits of highly stretchable elastomers have aided a myriad of modern technologies, including biomedical devices, flexible electronics, and soft robotics. However, the scope of their application to tissue engineering and regenerative medicine remains limited. The challenge is often that the seeded cells are exposed to substantial mechanical loads synchronized with the material, which causes undesirable effects in the cells as a result of them undergoing large deformations, even though the supporting scaffold is capable of withstanding them. Indeed, previous studies have shown that the seeded cells in such tough double-network hydrogels undergo elongation when stretched, and the corresponding severe strains experienced by the seeded cells can induce significant consequences such as nuclear envelope rupture, DNA damage, and apoptosis. Therefore, considering only the stretchability of the material itself in designing an artificial functional microtissue is not sufficient.

In some aspects, provided herein are scaffolds that protect cells from severe mechanical loads. In some embodiments, the scaffolds are flexible enough to conform with a host's native tissue (i.e., a target tissue). In some embodiments, the scaffolds have the unique property of mimicking multiple features of native tissue (i.e., target tissue), including flexibility, elasticity, and degradability. In some aspects, the scaffolds disclosed herein mimic the ability of native tissue (i.e., target tissue) to uncrimp under applied strain through the substrates' inherent self-pleating, rather than immediately stretch. In some embodiments, the properties of the scaffold are fine-tuned to match the parameters of a native tissue (i.e., target tissue), providing a unique and versatile platform for improved tissue generation.

In the past, knit materials have been used as a secondary skeleton to support collagen, fibrin, or other hydrogel-based substrates which host living cells. The scaffold used in these applications are not meant to be stretchable or to leverage their self-pleating designs, rather the opposite: they were designed to add structural support to a relatively weak framework where cells can easily survive and differentiate. Collagen and fibrin are adept at cultivating cells due to their natural biocompatibility, but the materials possess limited native strength—thus requiring the underlying skeleton of a knit or other stiff material. Alternatively, certain types of hydrogels are designed to be tough, stretchable, and biocompatible with adequate preprocessing and treatment. However, hydrogels do not possess native porosity typically needed for the integration with native tissue (i.e., target tissue) during healing. Furthermore, although hydrogels possess high stretchability, seeded cells stretch with them, which leads to increased cell death.

In contrast to previously used scaffolds, some aspects described herein relate to the use of knitted materials as the scaffold itself rather than as a secondary support skeleton for traditional scaffold materials. In some embodiments, the knit scaffolds disclosed herein enable high levels of cell survivability compared to other cell scaffolds, as the knits better protect seeded cells through their ability to uncrimp through a self-pleated architecture, rather than stretch. In some embodiments, the uncrimping of the knit scaffold causes minimal stretching of the cells. Additionally, in some embodiments, the knit scaffold has relevant design parameters that are tuned to: (a) match the natural elasticity of various native tissues (i.e., target tissues), and/or (b) control the degradation rate of the scaffolds to match applications requiring faster or slower healing times. Thus, in some embodiments, the knit scaffolds described herein comprise or consist of the entire scaffold, and are not part of a scaffold having multiple non-knit parts, such as collagen, fibrin, or hydrogel parts. In some embodiments, however, the knit scaffold may comprise an additional substrate which is added prior to cells being seeded on the scaffold. In some embodiments, an additional substrate is collagen. In some embodiments, the collagen is Type I, Type II, Type III, Type IV, and/or Type V collagen. In some embodiments, the additional substrate is hydrogel. In some embodiments, the additional substrate is fibrin.

### Knit Scaffold Structure

In some aspects, provided herein are three-dimensional knit scaffolds comprising multifilament biocompatible fibers arranged in a knit structure or design with intrinsic porosity, wherein the scaffold has an elasticity, stretchability, strain region of uncrimping, and anisotropy value of a native tissue (i.e., a target tissue). As used herein, a “native tissue” or “target tissue” refer to a tissue that is being targeted for tissue healing. Non-limiting examples of target tissues include brain, adipose, thymus, pancreas, kidney, eye (e.g., lens, cornea, and/or iris), liver, lung, spleen, thyroid, muscle, skin, bladder, gut, nerve, cartilage, ligament, and tendon. In some embodiments, the target tissue is located inside of a subject. In some embodiments, a subject is a human subject. In some embodiments, a human subject is in need of tissue healing due to an injury. In some embodiments, the three-dimensional knit scaffold comprises multifilament yarns knitted, sized, and shaped to support cells (e.g., promote cell growth). In some embodiments, the three-dimensional knit scaffold is used for tissue repair. In some embodiments, the size and/or shape of the knit scaffold depends on the intended use of the scaffold. In some embodiments, because the tissue scaffold provides mechanical support for the survival and/or growth of cells, such that the scaffold can be used to repair or replace tissues or parts of tissue, the size and/or shape of the device varies depending on the cell type and/or tissue type as well as the portion of tissue needing to be repaired or replaced.

Thus, in some embodiments, the three-dimensional knit scaffold has various dimensions, shapes, and/or properties, which match or closely mimic the shapes and/or mechanical properties of tissues in need of reconstruction, recovery, and/or regeneration (i.e., “target tissue”). In order to mimic these properties, in some embodiments particular properties of the knit scaffold are designed and chosen to have properties matching that of the target tissue. These properties may be, but are not limited to, knit structure, knit type, stitch length, number of filament ends plied together in a single yarn, filament diameter, filament cross sectional shape, yarn texture, filament material, custom shaping and/or tubular structures of knit fabric (e.g. knitted glove or sock pattern). As used herein, “knit structure” refers to the structure of the knit scaffold, e.g., the structure of the knit scaffold comprising any optional components, such as, for example, collagen, cells, etc.

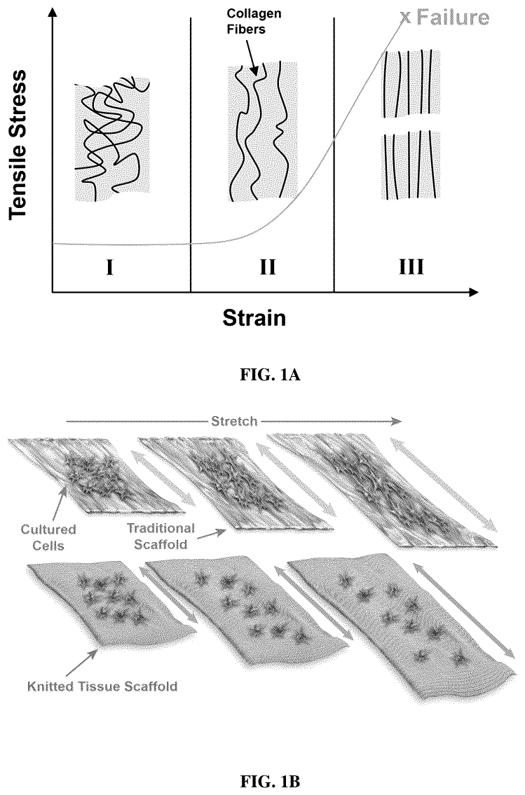

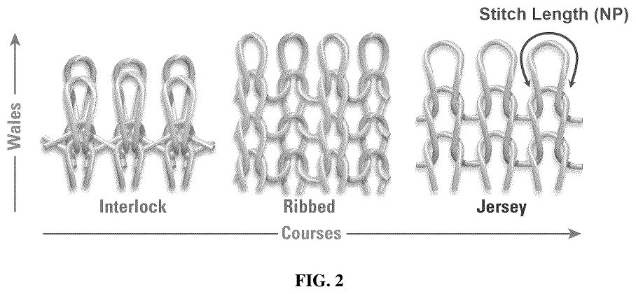

The knit type, as used herein, refers to the pattern created by the interconnecting fibers. In some embodiments, the knit type may be, but is not limited to, interlock, ribbed, or jersey, as shown in FIG. 2. In some embodiments, the knit fabric shape may be a flat, tubular, or custom shaped (e.g., shaped to match a body part). In some embodiments, the knit type used in the knit scaffold is designed based upon the desired stretchability (i.e., elasticity), ultimate strength, size, degradation rate, porosity, and/or level of uncrimp before stretching of the knit scaffold, in order to match the properties of the host tissue. As shown in FIG. 1A, natural tissue possesses a “level of uncrimp before stretching” as indicated in Region I; this is followed by a “transition region” (Region II) into a stretchable Region III wherein native Collagen fibers are elongated and begin to stretch. In some embodiments, these three properties of tissue (i.e., level of uncrimp before stretching [Region I], transition region [Region II], and stretchable region [Region III]) are replicated with variable weight as disclosed herein. In some embodiments, the knit scaffold may be comprised of multiple knit types in a single device. For example, a knit scaffold may be comprised of one-half interlock and one-half ribbed knits, one-half interlock and one-half jersey knits, or one-half ribbed and one-half jersey knits. In another example, the knit scaffold may be comprised of optionally alternating interlock, ribbed, and/or jersey knits. In another example, the knit scaffold may be comprised of different knit stitches to create a novel knit type. In some embodiments, for example, muscle with a Region III elasticity of nominally 90 MPa may be replicated with a knit scaffold made from PLA in a Jersey-type knit, with a stitch length controlled by a stitch cam setting on a Stoll knitting machine of 13 NP, and 4 ends of 150 denier air textured yarn with 64 filaments plied together into a single plied yarn. In another embodiment, tissue from the surface of a human bladder with a Region III elasticity of nominally 100-200 MPa may be replicated with a scaffold made from PLA in an Interlock-type knit, with a stitch length controlled by a stitch cam setting on a Stoll knitting machine of 13 NP, and 4 ends of 150 denier air textured yarn with 64 filaments plied together into a single plied yarn.

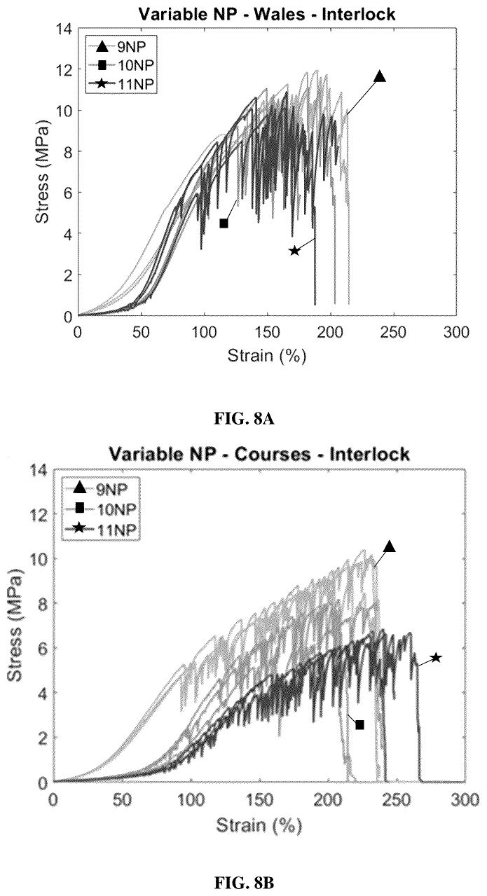

The stitch length, as used herein, refers to the length of yarn in a single knitted stitch from the position of the beginning of the loop to halfway between the current needle loop and the adjacent loop, as shown in FIG. 2. In some embodiments the stitch length may be, but is not limited to, 1 mm to 25 mm. In some embodiments the stitch length may be, but is not limited to, 3 mm to 15 mm. For example, the stitch length may be 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm. In some embodiments, altering the stitch length changes the length of the knit in the wales direction, courses direction, and/or in both directions. Altering the stitch length primarily affects the compliance of the knits in the courses direction as shown in FIG. 8; which can be used to tune elasticity and anisotropy to match host tissue. In some embodiments, multiple stitch lengths may be used in the knit of a single device; these may also be combined with different knit types. For example, a jersey knit with a stitch length of 10 mm may be combined with an interlock knit with a stitch length of 8 mm within a single scaffold device. In some embodiments, the stitch length is chosen based upon the desired stretchability (i.e., elasticity), strength, size, degradation rate, porosity, and/or level of uncrimp before stretching of the knit scaffold, in order to match the biomechanical properties of the host tissue.

The knit scaffold is comprised on interlocking biocompatible fibers in a knit sequence. A knit sequence, as used herein, refers to a set of fibers interlocking one another and having a defined knit type and stitch length. The knit sequence may define the entire knit scaffold or a portion of the knit scaffold. In some embodiments the entire knit scaffold has one knit sequence. In other embodiments the scaffold is comprised of multiple knit sequences, wherein the multiple knit sequences are different from one another, for instance, in knit type and/or stitch length. The scaffold may in some embodiments have multiple regions, some having different knit sequences and some having the same knit sequence. In some embodiments the scaffold has 2, 3, 4, 5, 6, 7, 8, 9, 10, 11, 12, 13, 14, 15, 16, 17, 18, 19, 20, 21, 22, 23, 24, or 25 knit sequences. In some embodiments the scaffold has 1-1000, 1-900, 1-800, 1-700, 1-600, 1-500, 1-400, 1-300, 1-200, 1-100, 1-95, 1-90, 1-85, 1-80, 1-75, 1-70, 1-65, 1-60, 1-55, 1-50, 1-45, 1-40, 1-35, 1-30, 1-25, 1-20, 1-15, 1-10, 1-5, 950-1000, 900-1000, 850-1000, 800-1000, 750-1000, 700-1000, 650-1000, 600-1000, 550-1000, 500-1000, 450-1000, 400-1000, 350-1000, 300-1000, 250-1000, 200-1000, 150-1000, 100-1000, 50-1000, 50-950, 100-900, 150-850, 200-800, 250-750, 300-700, 350-650, 400-600, 450-550, 475-525, 5-10, 10-15, 15-20, 20-25, 25-30, 30-40, 40-50, 50-60, 60-70, 70-80, 80-90, 90-100, 100-110, 110-120, 120-130, 130-140, 140-150, 150-160, 160-170, 170-180, 180-190, 190-200, 200-220, 220-240, 240-260, 260-280, 280-300, 300-325, 325-350, 350-375, 375-400, 400-425, 425-450, 450-475, 475-500, 500-550, 550-600, 600-650, 650-700, 700-750, 750-800, 800-850, 850-900, 900-950, or 950-1000 knit sequences.

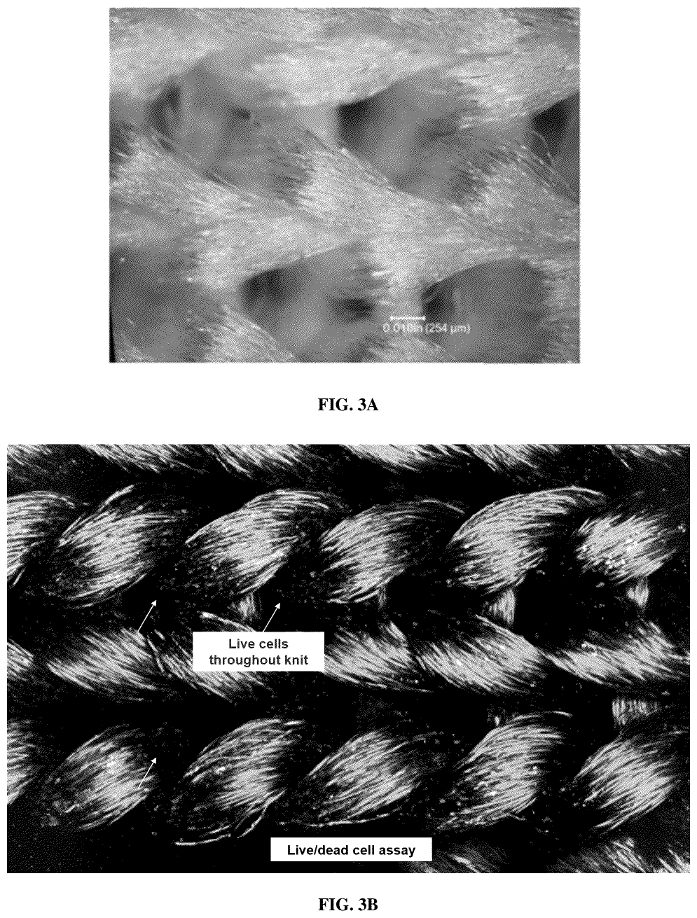

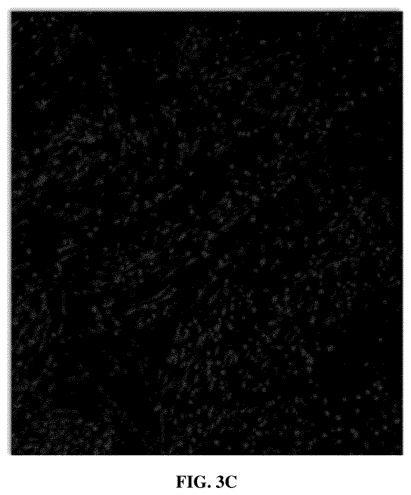

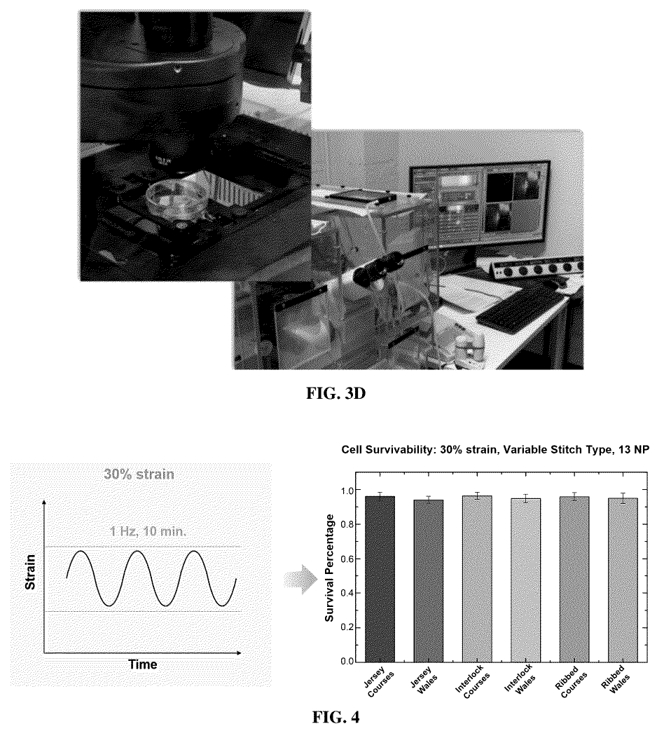

A biocompatible fiber, as used herein, is a thread or yarn (used interchangeably herein) comprising a polymeric or natural (e.g. silk) material. In some embodiments, the yarn comprises monomer and/or natural monofilaments, polymer and/or natural filaments, and/or copolymer and/or natural filaments. As used herein, monofilament yarns comprise of a single filament. Polymer filaments comprise more than one filament per yarn, wherein the filaments are comprised of the same material. FIGS. 3A-3C exemplify polymer filament yarn wherein the multiple filaments are made from PLA. Copolymer filaments are filaments that are comprised of one or more of materials. In some embodiments, the yarn is comprised of only monofilaments, only polymer filaments, only copolymer filaments, or only natural filaments. In some embodiments, the yarn is comprised of monofilaments and polymer filaments, monofilaments and copolymer filaments, monofilaments and natural filaments, polymer filaments and copolymer filaments, polymer filaments and natural filaments, or copolymer filaments and natural filaments. In other embodiments, the yarn is comprised of monofilaments, polymer filaments, and copolymer filaments. For example, a single yarn may be comprised of multiple filaments, half of which are made of PLA, and half of which are made of PGA. In another example, a single yarn may be comprised of multiple copolymer (or multicomponent) filaments, with the core of the filament made of PLA surrounded by a layer (or outer sheath) of PGA. In some embodiments, number of polymer and/or copolymer filaments per yarn varies, and in some embodiments are chosen based upon the desired stretchability (i.e., elasticity), ultimate strength, size, degradation rate, porosity, and/or level of uncrimp before stretching of the knit scaffold, in order to match the properties of the target tissue. In some embodiments, number of polymer and/or copolymer filaments per yarn is, but is not limited to, f2-f500. For example, a strand of yarn disclosed herein may comprise f5, f48, f64, f100, f150, f200, f250, f300, or f500 filaments. In some embodiments, a strand of yarn has f5-f480, f5-f460, f5-f440, f5-f420, f5-f400, f5-f375, f5-f350, f5-f325, f5-f300, f5-f280, f5-f260, f5-f240, f5-f220, f5-f200, f5-f190, f5-f180, f5-f170, f5-f160, f5-f150, f5-f140, f5-f130, f5-f120, f5-f110, f5-f100, f5-f95, f5-f90, f5-f85, f5-f80, f5-f75, f5-f70, f5-f65, f5-f60, f5-f55, f5-f50, f5-f45, f5-f40, f5-f35, f5-f30, f5-f25, f5-f20, f5-f15, f5-f10, f50-f500, f50-f480, f50-f460, f50-f440, f50-f420, f50-f400, f50-f380, f50-f360, f50-f340, f50-f320, f50-f300, f50-f280, f50-f260, f50-f240, f50-f220, f50-f200, f50-f190, f50-f180, f50-f170, f50-f160, f50-f150, f50-f140, f50-f130, f50-f120, f50-f110, f50-f100, f50-f90, f50-f80, f50-f70, f50-f60, f100-f500, f100-f480, f100-f460, f100-f440, f100-f420, f100-f400, f100-f380, f100-f360, f100-f340, f100-f320, f100-f300, f100-f280, f100-f260, f100-f240, f100-f220, f100-f200, f100-f190, f100-f180, f100-f170, f100-f160, f100-f150, f100-f140, f100-f130, f100-f120, f100-f110, f150-f500, f150-f480, f150-f460, f150-f440, f150-f420, f150-f400, f150-f380, f150-f360, f150-f340, f150-f320, f150-f300, f150-f280, f150-f260, f150-f240, f150-f220, f150-f200, f150-f190, f150-f180, f150-f170, f150-f160, or f200-f300 filaments.

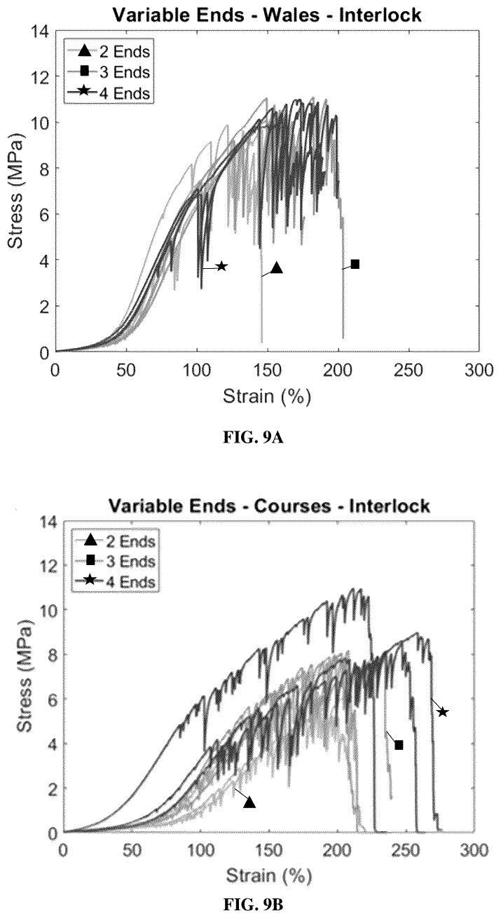

In some embodiments, filaments are plied (e.g., twisted, running parallel, joined, or fused) together through methods known in the art. In some embodiments, methods used to join multiple filaments together comprise, but are not limited to, heat fusion, twisting, braiding, blending, entangled, crimped, running parallel, carding and/or textured. The monofilaments, polymer filaments, and/or copolymer filaments may be plied together in to one or more “ends.” These ends may then be twisted together into the thicker yarns used to construct knit scaffolds. For example, the yarns used herein may be comprised of 1, 2, 3, 4, 5, 6, 7, 8, 9, 10 or more ends to add stiffness, increase ultimate strength, change porosity, and/or adjust other properties that may aid in regenerating tissue. In some embodiments, a yarn comprises between 1-5, between 5-10, between 10-15, between 15-20, between 20-25, between 25-30, between 30-35, between 35-40, between 40-45, between 45-50, between 1-3, between 1-4, between 1-5, between 1-6, between 1-7, between 1-8, between 1-9, between 1-10, between 1-11, between 1-12, between 1-13, between 1-14, between 1-15, between 1-16, between 1-17, between 1-18, between 1-19, between 1-20, between 1-22, between 1-24, between 1-26, between 1-28, between 1-30, between 1-33, between 1-36, between 1-38, between 1-40, between 1-42, between 1-44, between 1-46, between 1-48, between 1-50, between 10-20, between 20-30, between 30-40, between 40-50, between 5-15, between 15-25, between 25-35, between 35-45, between 45-55, or more ends. FIG. 9 demonstrates that in some embodiments, the number of ends adjusts the ultimate strength of the knits, with some additional degree of tunable elasticity in the courses direction.

In some embodiments, the scaffold is comprised of fibers or yarns having different biocompatible materials from one another. In some embodiments, the scaffold is comprised of fibers or yarns having the same biocompatible materials from one another. The biocompatible materials of the fibers may define the entire knit scaffold or a portion of the knit scaffold. In some embodiments the entire knit scaffold is comprised one type of biocompatible material. In other embodiments the scaffold is comprised of multiple different types of biocompatible materials. The scaffold may in some embodiments have multiple regions, each region comprised of a different biocompatible material.

In some embodiments, the biocompatible fibers disclosed herein for use in the knit scaffolds have a defined denier. A denier is a measurement used to define the size of a particular yarn or filament by measuring the linear density of the fibers in terms of grams per 9000 m made from biocompatible material or of the scaffold. The denier of the yarns or filaments disclosed herein may be, but is not limited to, 5-600. For example, in some embodiments, a yarn disclosed herein has a denier of 5, 10, 50, 100, 200, 300, 400, 500, or 600. In some embodiments, a yarn has a denier of between 1-5, between 5-10, between 10-15, between 15-20, between 20-25, between 25-30, between 30-35, between 35-40, between 40-45, between 45-50, between 50-55, between 55-60, between 60-65, between 65-70, between 70-75, between 75-80, between 80-85, between 85-90, between 90-95, between 95-100, between 100-110, between 110-120, between 120-130, between 130-140, between 140-150, between 150-160, between 160-170, between 170-180, between 180-190, between 190-200, between 200-220, between 220-240, between 240-260, between 260-280, between 280-300, between 300-325, between 325-350, between 350-375, between 375-400, between 400-425, between 425-450, between 450-475, between 475-500, between 500-550, between 550-600, between 1-10, between 1-15, between 1-20, between 1-25, between 1-30, between 1-35, between 1-40, between 1-45, between 1-50, between 1-55, between 1-60, between 1-65, between 1-70, between 1-75, between 1-80, between 1-85, between 1-90, between 1-95, between 1-100, between 1-110, between 1-120, between 1-130, between 1-140-, between 1-150, between 1-160, between 1-170, between 1-180, between 1-190, between 1-200, between 1-220, between 1-240, between 1-260, between 1-280, between 1-300, between 1-320, between 1-340, between 1-360, between 1-380, between 1-400, between 1-420, between 1-440, between 1-460, between 1-480, between 1-500, between 500-500, between 550-600, or more. In some embodiments, a yarn comprises filaments that have a denier of between 1-5, between 5-10, between 10-15, between 15-20, between 20-25, between 25-30, between 35-40, between 40-45, between 45-50, between 50-55, between 55-60, between 60-65, between 65-70, between 70-75, between 75-80, between 80-85, between 85-90, between 90-95, between 95-100, between 10-20, between 20-30, between 30-40, between 40-50, between 50-60, between 60-70, between 70-80, between 80-90, between 90-100, between 20-40, between 40-60, between 60-80, between 80-100, between 1-10, between 1-15, between 1-20, between 1-25, between 1-30, between 1-35, between 1-40, between 1-45, between 1-50, between 1-55, between 1-60, between 1-65, between 1-70, between 1-75, between 1-80, between 1-85, between 1-90, between 1-95, between 1-100 or more. In some embodiments, the filament denier is based upon the desired stretchability (i.e., elasticity), ultimate strength, size, degradation rate, and/or level of uncrimp before stretching of the knit scaffold, in order to match the properties of the host tissue. In some embodiments, the denier can vary between filaments within a single strand of yarn, or between strands of yarn within a knit scaffold. For example, a biocompatible yarn may be comprised of filaments with deniers of 5, 50, and 100, which are plied together to create a single strand of yarn.

In some embodiments, the biocompatible fibers are designed to exhibit a desired stretchability (i.e., elasticity), ultimate strength, size, degradation rate, and/or level of uncrimp before stretching of the knit scaffold, in order to match the properties of the host tissue. The material of the knitted filaments may be, but is not limited to, polyhydroxy acids such as polylactic and polyglycolic acids or blends thereof, including polylactic acid (PLA), polyglycolic acid (PGA), polylactic-co-glycolic acid (PLGA), poly-l-lactic acid (PLLA), poly-d-lactic acid (PLDA), polyanhydrides, polyorthoesters, polyphosphazenes, polycaprolactones, biodegradable polyurethanes, polyanhydride-co-imides, polypropylene fumarates, polydiaxonane, polyhydroxyalkanoate, poly(trimethylene carbonate), and/or combinations thereof in the form of blends and copolymers. Suitable polyhydroxyal kanoate homopolymers include poly-3-hydroxybutyrate (PHB), poly-4-hydroxybutyrate (P4HB), poly-3-hydroxyvalerate (PHV), poly-3-hydroxypropionate (PHP), poly-2-hydroxybutyrate (P2HB), poly-4-hydroxyvalerate (P4HV), poly-5-hydroxyvalerate (P5HV), poly-3-hydroxyhexanoate (PHH), poly-3-hydroxyoctanoate (PHO), poly-3-hydroxyphenylvaleric acid (PHPV) and poly-3-hydroxyphenylhexanoic acid (PHPH). In alternative embodiments, polyhydroxy alkanoate copolymers including poly(3-hydroxybutyrate co-3-hydroxyvalerate) (PHBV) and poly(3-hydroxybutyrate-co-3-hydroxyhexanoate) (PHBH). Additionally, the filaments made be made from Polydioxinone (PDS or PDO), which may also potentially be combined in various combinations with other polyhydroxy acids mentioned previously. Silk may also be used solely as a biocompatible fiber or may also potentially be combined in various combinations with other polymers mentioned previously.

The biocompatible filaments are typically biodegradable (i.e., bioresorbable). Natural biodegradable polymers such as proteins and polysaccharides, for example, extracellular matrix components, collagen, fibrin, polysaccharide, cellulose, silk, or chitosan, may also be used.

Biodegradable materials and/or non-biodegradable materials may be used. The classification of biodegradable and non-biodegradable materials is not absolute. For example, most polyesters are non-biodegradable (such as polyethylene terephthalate), except that some polyesters (such as those made from polyglycolic acid, polylactic acid, or polyhydroxyalkanoates) are biodegradable. Similarly, silk is generally considered as a non-biodegradable material, but over a long period of time (e.g., 10 to 25 years), the body can break down silk implanted in the body, or the silk can be modified to be more biodegradable.

The tissue scaffolds disclosed herein have microscopic and macroscopic cell porosity. For microscopic porosity, the materials have sufficient space or texture to house cells and/or other biological materials. This is typically achieved with multifilament yarn whereby the cells live and grow on and/or in between filaments. This can also be achieved with monofilaments through texture and/or size. Additionally, the material of monofilaments may not require a specific microporosity, but a macroscopic porosity will still exist. When the microscopic porosity space is sized such that it is sufficient to house cells (for example by varying the size, shape, density, or overall number of filaments plied together in a yarn), the tissue scaffold has a minimal cell seeding porosity and maximum surface area to harvest cells. The macroscopic porosity refers to the larger spaces in between knitted structures (typically 100 micrometers-2 millimeters) and varies with stretching. The macroscopic porosity can be varied using the design parameters discussed herein (e.g., knit structure, knit type, stitch length, filament denier, number of filaments in a single “end”, number of ends plied together in a yarn, number of stitches per length, etc.) to enable the host tissue to grow within the knit scaffold. Macroscopic porosity is catered on an application-specific basis depending on, but not limited to, factors like recovery rate of the host tissue, hydraulic permeability of the tissue, hydraulic permeability of the scaffold, hydraulic permeability of the tissue and scaffold combination through various stages of recovery. The microscopic porosity, its corresponding minimal seeding porosity, and the macroscopic porosity will be determined by the type of cell(s) or biological materials to be seeded on the tissue scaffold in addition to characteristics of the host tissue.

### Tunable Knit Scaffold Devices

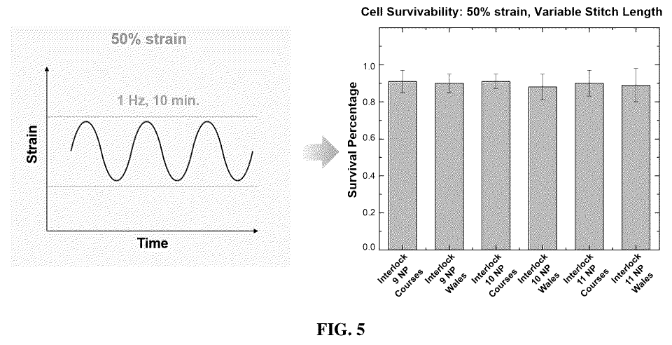

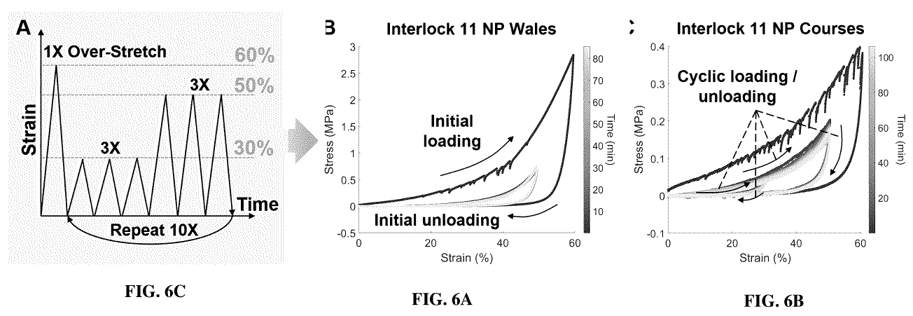

The knit scaffolds disclosed herein mimic the natural flexibility of human tissue, such that the knit scaffolds are capable of flexing while the embedded cells maintain high cell survivability. The ability of the knit scaffolds to mimic the natural ‘uncrimping’ effect of tissue through inherent self-pleating rather than stretching provides unique advantages to the cells growing within the scaffold and avoiding the local stretching of cells at the micro level. The knitted structures disclosed herein can be tuned mechanically to promote compatibility with various injuries and tissue types. The scaffold is “tuned” by manipulating the design parameters related to properties such as knit structure, knit type, stitch length, filament denier, number of filaments in an “end”, number of ends plied together in a single yarn, and type of biocompatible material in order to produce a scaffold having a particular or a range of elasticity (which applies to Regions I and III as shown in FIG. 1A), range of “uncrimping” region (i.e., Region I as shown in FIG. 1A, and the radius of the curve in Region II) stretchability, and anisotropy values. FIGS. 5-10, collectively, demonstrate that these properties can be tuned and additionally, that an initial over-stretch or repeated stretch of the fabric will create more compliant and repeatable properties thereafter. After initial loading, the knitted structures maintain a repeatable elasticity during loading and unloading as shown in FIG. 6.

The elasticity of the scaffold, as used herein refers to the ability of a deformation or stretch in the scaffold material to return to its original shape and size when the forces causing the deformation are removed. Young's modulus, also known as elastic modulus is the measure of elasticity of the scaffold material. Methods are known to the skilled artisan for assessing the elasticity of the fibers and/or knit scaffold. These methods utilize a variety of machines including, but not limited to, tensile testers, ball-burst strength testers, bi-axial strain testers and/or diaphragm-based bursting apparatuses. For instance, knits can be clamped in to the jaws of a tensile testing machine with integrated force transducers and displacement measurement sensor(s). The tested samples are typically measured in cross-sectional area before, and potentially during and after testing to determine the cross-sectional area change. Upon stretching the stress of the sample as defined by the measured force divided by the cross-sectional area is recorded simultaneously with the displacement of the sample. The strain is then calculated as the ratio of the change in displacement subtracted from the initial displacement divided by the initial displacement. In this fashion the elasticity of a sample in Regions I, II, and/or III from FIG. 1A is defined as the slope of the curve when stress is plotted against strain. Elasticity of the scaffolds disclosed herein may refer to any region of the stress strain curve (i.e., Region I, Region II, and/or Region III). A common commercial supplier for machines that automate this process is Instron®. Other spatially-sensitive methods for tracking local strain of the scaffolds may be implemented including, but not limited to, Digital Image Correlation (DIC), Ultrasound Elasticity Imaging (UEI), or microrheology.

The stretchability of the scaffold, as used herein refers to the ability of the material comprising the scaffold to be stretched, as opposed to ‘uncrimped’, through regions I, II, and/or III as shown in FIG. 1A. In order to tune the scaffold based on stretchability, the type of biocompatible material(s) used to produce the fibers may be varied. Additionally, the design of the knit scaffold may be varied by changing parameters including, but not limited to, filament denier, number of filaments within and “end”, number of ends plied together in a yarn, stitch length, and knit type. The present disclosure embraces the natural ability to uncrimp and the natural stretchability of knits themselves (i.e., the elasticity of Regions I and III, respectively from FIG. 1A, and the size and radius of the curve between them in Region II). The present disclosure generally relates to knit scaffolds. Rather than using a stiff knit that does not stretch, the inherent ability of a knit to uncrimp is leveraged to allow for scaffold movement, such that the knit scaffold has uncrimping or movement capabilities while protecting the cells seeded on the knit scaffold from stretching on a micro level. Stretchability of biocompatible materials that compose the yarn that makes the knits is known to the skilled artisan. Stretchability of a macroscopic knit is also known qualitatively in complement to the quantitative elasticity to the skilled artisan.

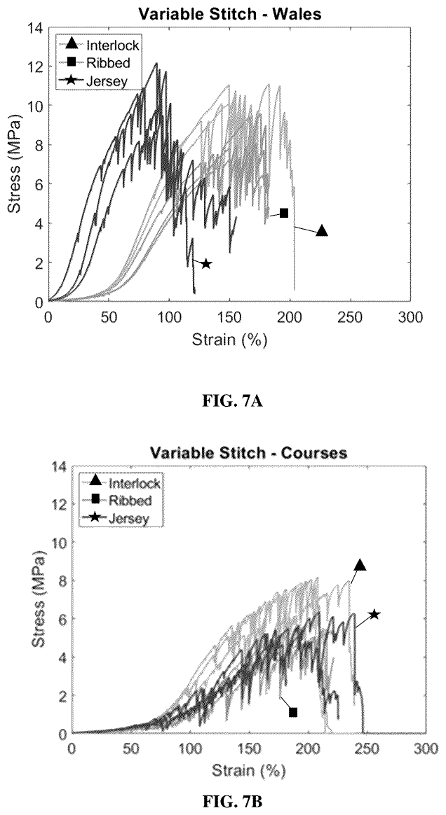

The anisotropy of the scaffold, as used herein refers to the ability of the scaffold to have variable elasticity in multiple directions. The anisotropic properties of different tissues are known. The knit scaffolds are designed to mimic the anisotropic properties of different tissues. The anisotropy of a knit is generally characterized by testing the same or potentially different knits in orthogonal directions to measure elasticity as defined previously. Additionally, the diagonals of the material can be tested, for instance to measure the 45 degree “bias” angle of the knits. The anisotropy may be defined by testing any of the Regions I, II, and/or III as indicated in FIG. 1A and leveraged to mimic a particular type of tissue in its same characteristic region. Anisotropy can be varied by changing design parameters of the knits including, but not limited to, filament denier, number of filaments within and “end”, number of ends plied together in a yarn, stitch length, and knit type. Additional courses or wales-specific yarns may be added in addition to the base knit to change its elasticity in one direction vs. the other orthogonal direction. Anisotropy can all be varied by varying the knit, purl, tuck, or skip stitch that makes up a knitted structure on the front and back beds of a knitting machine. FIGS. 7 and 8 demonstrate that knit type and stitch length can adjust the anisotropy in orthogonal directions. Anisotropy can be measured by testing a scaffold of the same design in orthogonal directions sequentially; or, additionally, it can be measured simultaneously on a biaxial testing machine. The geometric anisotropy can also be measured to characterize a scaffold which is defined as the change in width or length while the orthogonal direction, respectively, is being stretched.

The ultimate strength of the scaffold as used herein refers to the maximum stress or force that the scaffold can withstand before failure. In some embodiments, ultimate strength of a scaffold is calculated by plotting a stress-strain curve.

In some embodiments, a knit scaffold is “primed” prior to being used in a target tissue. As used herein, the term “primed” refers to an initial disturbance of the knit scaffold such that following the disturbance, the knit scaffold exhibits repeatable parameters (e.g., repeatable measurements of a stress/strain curve). Taken as an example, FIGS. 6B and 6C show readings of knits that were cyclically strained. The results from the profile of FIG. 6A were plotted as a function of stress, strain, and time. FIGS. 6B and 6C demonstrate that the initial stretch results in intermittent and sharp variations in response as the knit re-distributes stress in to a more uniform and balanced state. Upon subsequent loading to 30% and 50%, the cyclic loading/unloading curve is repeatable with overall lower values of stress per strain. The initial over-stretching of the scaffold followed by the repeatable measurements thereafter, as shown in FIG. 6, demonstrates an example of an initial priming of the scaffold. Thus, in some embodiments, a knit scaffold is primed by over-stretching the scaffold. A scaffold is said to be over-stretched if it is stretched beyond the amount it would be stretched during its intended use. For example, a knit scaffold designed to undergo about 50% strain in a target tissue would be considered to be over-stretched if the scaffold were strained more than 50%. In some embodiments, a knit scaffold is over-stretched by 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 220%, 240%, 260%, 280%, 300%, or more. In some embodiments, a knit scaffold is over-stretched between 5-10%, between 10-15%, between 15-20%, between 20-25%, between 25-30%, between 30-35%, between 35-40%, between 40-45%, between 45-50%, between 50-55%, between 55-60%, between 60-65%, between 65-70%, between 70-75%, between 75-80%, between 80-85%, between 85-90%, between 90-95%, between 95-100%, between 100-110%, between 110-120%, between 120-130%, between 130-140%, between 140-150%, between 150-160%, between 160-170%, between 170-180%, between 180-190%, between 190-200%, between 200-220%, between 220-240%, between 240-260%, between 260-280%, between 280-300%, between 1-5%, between 1-10%, between 1-15%, between 1-20%, between 1-25%, between 1-30%, between 1-35%, between 1-40%, between 1-45%, between 1-50%, between 1-55%, between 1-60%, between 1-65%, between 1-70%, between 1-75%, between 1-80%, between 1-85%, between 1-90%, between 1-95%, between 1-100%, between 1-110%, between 1-120%, between 1-130%, between 1-140%, between 1-150%, between 1-160%, between 1-170%, between 1-180%, between 1-190%, between 1-200%, between 1-220%, between 1-240%, between 1-260%, between 1-280%, between 1-300%, between 50-100%, between 100-150%, between 150-200%, between 200-250%, between 250-300%, or more. In some embodiments, a knit scaffold is primed through cyclic stretching of the scaffold. In some embodiments, a knit scaffold is primed by applying heat to the scaffold. In some embodiments, the scaffold is heated to between 35-100° C., 40-100° C., 45-100° C., 50-100° C., 55-100° C., 60-100° C., 65-100° C., 70-100° C., 75-100° C., 80-100° C., 85-100° C., 90-100° C., 95-100° C., 35-40° C., 35-45° C., 35-50° C., 35-55° C., 35-60° C., 35-65° C., 35-70° C., 35-75° C., 35-80° C., 35-85° C., 35-90° C., 35-95° C., 40-45° C., 40-55° C., 40-60° C., 40-65° C., 40-70° C., 40-75° C., 40-80° C., 40-85° C., 40-90° C., 40-95° C., 40-100° C., 45-55° C., 45-60° C., 45-65° C., 45-70° C., 45-75° C., 45-80° C., 45-85° C., 45-90° C., 45-95° C., 45-100° C., 50-60° C., 50-65° C., 50-70° C., 50-75° C., 50-80° C., 50-85° C., 50-90° C., 50-95° C., 50-100° C., 55-65° C., 55-70° C., 55-75° C., 55-80° C., 55-85° C., 55-90° C., 55-95° C., 55-100° C., 60-70° C., 60-75° C., 60-80° C., 60-85° C., 60-90° C., 60-95° C., 60-100° C., 65-75° C., 65-80° C., 65-85° C., 65-90° C., 65-95° C., 65-100° C., 70-80° C., 70-85° C., 70-90° C., 70-95° C., 70-100° C., 75-85° C., 75-90° C., 75-95° C., 75-100° C., 80-90° C., 80-95° C., 80-100° C., 85-95° C., 85-100° C., 90-100° C., or more than 100° C. In some embodiments, a knit scaffold is primed by heating the knit scaffold to a temperature that is increased from a baseline temperature by 5%, 10%, 15%, 20%, 25%, 30%, 35%, 40%, 45%, 50%, 55%, 60%, 65%, 70%, 75%, 80%, 85%, 90%, 95%, 100%, or more. In some embodiments, a knit scaffold is primed by heating the knit scaffold to a temperature that is increased from a baseline temperature between 5-10%, between 10-15%, between 15-20%, between 20-25%, between 25-30%, between 30-35%, between 35-40%, between 40-45%, between 45-50%, between 50-55%, between 55-60%, between 60-65%, between 65-70%, between 70-75%, between 75-80%, between 80-85%, between 85-90%, between 90-95%, between 95-100%, between 1-5%, between 1-10%, between 1-15%, between 1-20%, between 1-25%, between 1-30%, between 1-35%, between 1-40%, between 1-45%, between 1-50%, between 1-55%, between 1-60%, between 1-65%, between 1-70%, between 1-75%, between 1-80%, between 1-85%, between 1-90%, between 1-95%, between 1-100%, between 5-15%, between 15-25%, between 25-35%, between 35-45%, between 45-55%, between 55-65%, between 65-75%, between 75-85%, between 85-95%, between 20-40%, between 40-60%, between 60-80%, between 80-100%, or more. As used herein, a baseline temperature refers to the temperature at which the knit scaffold is designed to be used at (e.g., a temperature of a target tissue). In some embodiments, a knit scaffold is primed through a combination of over-stretching, cyclic stretching, and/or heating of the scaffold.

In some embodiments, the knit scaffold is used for skin generation or regeneration. A knit scaffold for skin generation has properties that mimic normal skin tissue, such as human skin tissue. For instance, in some embodiments, a knit scaffold for skin generation has a skin elasticity value of between 5 Pa-10 MPa and knit an anisotropy value ranging from between 1:1.01 to 1:10.

In some embodiments, the knit scaffold is used for ligament generation. In some embodiments, a knit scaffold for ligament generation has properties that mimic normal ligament tissue, such as human ligament tissue. For instance, in some embodiments, a knit scaffold for ligament generation has a ligament elasticity value of between 1 MPa to 10 GPa and an anisotropy value of between 1.01 to 1:10.

In some embodiments, the knit scaffold is used for muscle generation. In some embodiments, knit scaffold for muscle generation has properties that mimic normal muscle tissue, such as human muscle tissue. For instance, in some embodiments, a knit scaffold for muscle generation has a muscle elasticity value of between 1 kPa to 10 MPa and an anisotropy value of between 1:1.01 to 1:10.

In some embodiments, the knit scaffold is used for cartilage generation. In some embodiments, a knit scaffold for cartilage generation has properties that mimic normal cartilage tissue, such as human cartilage tissue. For instance, in some embodiments, a knit scaffold for cartilage generation has a cartilage elasticity value of between 1 kPa to 10 MPa and an anisotropy value of between 1.01 to 1:10.

In some embodiments, the knit scaffold is used for adipose (fatty tissue) generation. In some embodiments, a knit scaffold for adipose generation has properties that mimic normal adipose tissue, such as human adipose tissue. For instance, in some embodiments, a knit scaffold for adipose generation has an adipose elasticity value of between 1 Pa to 100 kPa and an anisotropy value of between 1.01 to 1:10.

In some embodiments, the knit scaffold is used for bone generation. In some embodiments, a knit scaffold for bone generation has properties that mimic normal bone properties, such as human bone. For instance, in some embodiments, a knit scaffold for bone generation has a bone elasticity value of between 10 MPa to 200 GPa and an anisotropy value of between 1.01 to 1:10.

For all the aforementioned non-limiting examples, in some embodiments, the range of biomechanical properties is chosen or designed to match Region I, Region II, or Region III (as based on FIG. 1A). In some embodiments, the variable biomechanical properties are achieved by changing parameters including, but not limited to, filament denier, number of filaments within and “end”, number of ends plied together in a yarn, stitch length, and knit type.

### Manufacture of Knitted Cell Scaffolds

In some embodiments, the scaffold is constructed on a fine gauge crochet knitting machine. A non-limiting list of crochet machines capable of manufacturing the knit scaffold according to aspects of the present disclosure are provided by: Comez; RIUS-Comatex; Changde Textile Machinery Co., Ltd.; China Textile Machinery Co., Ltd.; Huibang Machine; Jakob Muller AG; Jingwei Textile Machinery Co., Ltd.; Zhejiang Jingyi Textile Machinery Co., Ltd.; Dongguan Kyang the Delicate Machine Co., Ltd.; Karl Mayer; Sanfang Machine; Sino Techfull; Suzhou Huilong Textile Machinery Co., Ltd.; Taiwan Giu Chun Ind. Co., Ltd.; Zhangjiagang Victor Textile; Liba; Lucas; Muller Frick; and Texma.

In some embodiments, the scaffold is knitted on a fine gauge warp knitting machine. A non-limiting list of warp knitting machines capable of manufacturing the knit scaffold according to aspects disclosed herein are provided by: Comez; RIUS-Comatex; Diba; Jingwei Textile Machinery; Liba; Lucas; Karl Mayer; Muller Frick; Monarch Knitting Machinery Corporation; Runyuan Warp Knitting; Taiwan Giu Chun Ind.; Fujian Xingang Textile Machinery; and Yuejian Group.

In some embodiments, the scaffold is knitted on a fine gauge flat bed knitting machine. A non-limiting list of flat bed machines capable of manufacturing the knit scaffold are provided by: Stoll, Shima Seiki; Brother, Karl Meyer, Around Star; Boosan; Cixing Textile Machine; Fengshen; Flying Tiger; Machinery; Fujian Hongqi; G & P Gorteks; Jinlong; JP; Jy Leh; Kauo Heng Co., Ltd.; Matsuya; Nan Sing Machinery Limited; Nantong Sansi Instrument; Nantong Tianyuan; and Ningbo Yuren Knitting.

In some embodiments, the scaffold is knitted on a cylindrical knitting machine. A non-limiting list of flat bed machines capable of manufacturing the knit scaffold are provided by: Lonati S.p.A; Sintelli; Fukuhara Industrial & Trading Co., Ltd., Meyer & Cie GmbH & Co. KG, Orizio, Hanma group, Juinn Long Machine Co., Ltd., Zentex, Masa, Well, Jin Har, Pailung, Lisky, Tompkins USA, Terrot, and Santoni.

### Knitted Cell Scaffolds for Use in Tissue Recovery

### Seeding Cells for Growth on Knitted Cell Scaffolds

In some embodiments, the knit scaffold is seeded with cells, preferably mammalian cells, more preferably human cells. In a preferred embodiment, the human cells are allogeneic. Various cell types can be used for seeding. Non-limiting exemplary cell types which can be seeded into the knit scaffolds when used for reconstruction, regeneration or augmentation of connective tissue or other tissue types include chondrocytes, osteoblasts, fibroblasts, angioblasts, myoblasts, epithelial cells, urothelial cells, smooth muscle cells, keratinocytes, beta cells, endothelial cells, fibrocytes, vascular endothelial cells, hepatocytes, small intestine epithelial cells, epidermal keratinocytes, bone marrow mesenchymal cells, cardiomyocytes, immune cells, intervertebral disc cells, oral mucosal epithelia, gastrointestinal mucosal epithelia, urinary tract epithelia, skeletal joint synovium, periosteum, perichondrium, skeletal muscle cells, smooth muscle cells, cardiac muscle cells, pericardium, dura, meninges, keratinocyte precursor cells, pericytes, glial cells, neural cells, amniotic membrane, placental membrane, serosal cells, undifferentiated stem cells, undifferentiated progenitor cells, pre-differentiated stem cells, pre-differentiated progenitor cells, neural stem cells, neural progenitor cells, neuronal cells, dendritic cells, genetically engineered cells, and/or stem cells. Cells used may be harvested, grown and passaged in tissue cultures, they may be from a cultured cell line, or they may be from tissue harvested and dissociated at the time of grafting.

In some embodiments, pluri- or multi-potent stem cells are harvested from a subject in need of tissue recovery, and these stem cells are subsequently seeded on the knit scaffold prior to engrafting the scaffold in the subject. In some embodiments, stem cells are induced pluripotent stem cells (iPSCs), wherein allogeneic cells are first harvested from a host subject, then transformed into iPSCs using methods well-known in the art.

In some embodiments, before seeding cells onto the knit scaffold, a Human Leukocyte Antigen (HLA) typing test is performed to see if the host subject's body will reject the new cells. An HLA test evaluates the type of HLA proteins found in the host subject and compares them to the allogeneic cells to determine if the host subject's immune system will attack the new cells; methods for performing this test are well-known in the art. In some embodiments, if the HLA test result is negative (i.e., the host subject's immune system will not attack the grafted cells), the allogeneic cells are seeded onto the knit scaffold.

In some embodiments, cells are seeded and evaluated for health prior to seeding them on the knit scaffold. In some embodiments, the cells are evaluated for health through the following process: (1) the knit scaffolds are washed in ethanol, isopropyl alcohol and/or water. Optionally, the cells are then exposed to ultraviolet light for at least 2 hours. In some embodiments, the initial wash in water or alcohol is agitated through ultrasonic methods. In some embodiments, the wash medium may be replaced to wash the cells. In some embodiments, the wash medium is replaced 2-3 or more times. (2) the knit scaffolds are dried and seeded with either Embryonic Fibroblasts (EFs) or other types of stem cells. (3) the knit scaffolds with cells are cultured. In some embodiments, the knit scaffolds with cells are cultured using Dulbecco's Modified Eagle Medium (DMEM) medium supplemented with 10% Fetal Bovine Serum (FBS) and 1% penicillin streptomycin. (4) the knit scaffolds with the cells are imaged using a confocal microscope (e.g., a Leica SP8) as shown in FIGS. 3B-3D. In some embodiments, to assess cell viability, the cells are transfected to express a fluorescent protein (e.g., green fluorescent protein)-tagged nuclear localization signal which only localizes in cell nuclei when cells are alive (shown in FIGS. 3B-3C). (5) the number of live cells on the fabrics can be counted in post-processing.

In some embodiments, after washing in water or alcohol as described previously, the scaffolds are further sterilized in an Ethylene Oxide (EtO) chamber. In some embodiments, during this process the temperature is maintained between 68° F. (20° C.) and 91° F. (33° C.) for the full sterilization cycle. In some embodiments, humidity is maintained at nominally 35%. In some embodiments, the sterilization is run in 12- or 24-hour cycles. In some embodiments, the sterilization comprises a subsequent 24-hour aeration period before vacuum drying and packaging in sterile sealed bags. In some embodiments, the sterilization process may include gamma or E-beam exposure.

### Knit Scaffold Stability and Degradation

In some embodiments, the knit scaffold is fully or partly degradable over a defined period of time. In some embodiments, the biodegradation rate of the scaffolds depends on the chemical composition of the material and/or additional factors, such as, but not limited to, size, surface area-to-volume ratio, strength, elasticity, and contributions of the local environment, such as, but not limited to, mechanical forces, pH, exposure to water, cellular activity, mechanical wear, etc. In some embodiments, depending on the composition, knit structure, knit type, filament number per yarn, and/or denier of the filaments and yarns used in the knit scaffolds, the knit scaffold has varying degradation rates. In some embodiments, the stability of the knit scaffold is characterized by the length of the support period, transition period, and/or degradation period. In some embodiments, a knit scaffold has a support period and degradation period only, each with varying lengths (e.g., one week or three years), after which the scaffold is completely degraded. Other scaffolds may have a support period, transition period, and degradation period, each with various lengths, such as one week to three years or one to six months. In some embodiments, a knit scaffold has a support period, a transition period, and/or a degradation period of about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 22 days, 24 days, 26 days, 28 days, 30 days, 32 days, 34 days, 36 days, 38 days 40 days, 42 days, 44 days, 46 days, 48 days, 50 days, 55 days, 60 days, 65 days, 70 days, 75 days, 80 days, 85 days, 90 days, 95 days, 100 days, 110 days, 120 days, 130 days, 140 days, 150 days, 160 days, 170 days, 180 days, 190 days, 200 days, 220 days, 240 days, 260 days, 280 days, 300 days, 325 days, 350 days, 375 days, 400 days, 425 days, 450 days, 475 days, 500 days, 550 days, 600 days, 650 days, 700 days, 750 days, 800 days, 850 days, 900 days, 950 days, 1000 days, 1100 days, 1200 days, 1300 days, 1400 days, 1500 days, 1600 days, 1700 days, 1800 days, 1900 days, 2000 days, or more. In some embodiments, a knit scaffold has a support period, a transition period, and/or a degradation period of between 1-5 days, between 5-10 days, between 10-15 days, between 15-20 days, between 20-25 days, between 25-30 days, between 30-35 days, between 35-40 days, between 40-45 days, between 45-50 days, between 50-55 days, between 55-60 days, between 60-65 days, between 65-70 days, between 70-75 days, between 75-80 days, between 80-85 days, between 85-90 days, between 90-95 days, between 95-100 days, between 100-110 days, between 110-120 days, between 120-130 days, between 130-140 days, between 140-150 days, between 150-160 days, between 160-170 days, between 170-180 days, between 180-190 days, between 190-200 days, between 200-220 days, between 220-240 days, between 240-260 days, between 260-280 days, between 280-300 days, between 300-325 days, between 325-350 days, between 350-375 days, between 375-400 days, between 400-425 days, between 425-450 days, between 450-475 days, between 475-500 days, between 500-550 days, between 550-600 days, between 600-650 days, between 650-700 days, between 700-750 days, between 750-800 days, between 800-850 days, between 850-900 days, between 900-950 days, between 950-1000 days, between 1000-1100 days, between 1100-1200 days, between 1200-1300 days, between 1300-1400 days, between 1400-1500 days, between 1500-1600 days, between 1600-1700 days, between 1700-1800 days, between 1800-1900 days, between 1900-2000 days, or more.

In some embodiments, biodegradation of biomaterials involves cleavage of hydrolytically or enzymatically sensitive bonds in the filaments, leading to filament breakage. In some embodiments, the degradation rate of the scaffold is dependent on the degradation rate of the filaments and yarns used to form the scaffolds. For example, in some embodiments, rapidly degrading filaments and yarns lose between 80% and 90% of the polymer molecular weight within one week, two weeks, three weeks, one month, two months, three months, one year, two years, or more following implantation. In some embodiments, these filaments are entirely degraded during the support period of the scaffold, leaving behind gradually degrading, slowly degrading, and/or very slowly degrading filaments and yarns. Non-limiting examples of rapidly degrading filaments and yarns include filaments and yarns made from polyglycolic acid. In some embodiments, gradually degrading filaments and yarns lose between 5% and 10% of the molecular weight of the filament within the initial one week, two weeks, three weeks, one month, two months, three months, or more following grafting. In some embodiments, the gradually degrading filaments and yarns then lose between 10% and 80% of their molecular weight within the next one month, two months, or three months. In some embodiments, these gradually degrading filaments may be almost entirely degraded during the transition period of the scaffold, leaving behind slowly degrading and/or very slowly degrading filaments and yarns. In some embodiments, slowly degrading filaments and yarns lose between 0% and 5% of the molecular weight within the initial one week, two weeks, three weeks, one month, two months, three months, or more following implantation. In some embodiments, these slowly degrading filaments and yarns then lose between 5% and 20% of their structural integrity within the next one month, two months, three months, or more, then fully degrade within the following one month, two months, three months, or more. In some embodiments, these slowly degrading filaments may be almost entirely degraded during the degradation period of the scaffold, leaving behind very slowly degrading filaments, fibers, and yarns.

In some embodiments, very slowly degrading filaments and yarns remain stable during the initial one to three years of implantation and gradually degrade during next three to six years. Non-limiting examples of very slowly degrading filaments and yarns include filaments and yarns formed of polyethylene, polyether terephthalate, polystyrene, silicone, polytetrafluoroethylene, polyacrylic acid, a polyamide (e.g., nylon), polycarbonate, polysulfone, polyurethane, polybutadiene, polybutylene, polyethersulfone, polyetherimide, polyphenylene oxide, polymethylpentene, polyvinylchloride, polyvinylidene chloride, polyphthal amide, polyphenylene sulfide, polyetheretherketone (i.e., “PEEK”), polyimide, polymethylmethacylate polypropylene, and/or silk. In some cases, the material may include a ceramic such as tricalcium phosphate, hydroxyapatite, fluorapatite, aluminum oxide, or zirconium oxide, bioglass, and additional polymers, such as polyhydroxylethylmethacrylate (PHEMA) and poly(methyl methacrylate) (PMMA). In some embodiments, scaffold degradation occurs through mechanisms that involve physical and/or chemical processes, and/or biological processes mediated by biological agents, such as enzymes in tissue remodeling. The biodegradable scaffold gradually degrades by predetermined period to be replaced by newly grown tissue from the adhered cells. Degradation results in scaffold dismantling and material dissolution/resorption through the scaffolds' bulk and/or surface types of degradation. Scaffolds undergoing bulk degradation have a breakdown of the internal structure of the scaffold, thus reducing the molecular mass. In some embodiments, a scaffold that primarily undergoes surface degradation can be described similarly to the dissolution of soap. Generally, the rate at which the surface degrades is constant. Therefore, in some embodiments, while the size of the scaffold becomes smaller, the bulk structure is maintained. In some embodiments, these types of degrading scaffolds provide longer mechanical stability for the tissue to regenerate.

### Tissue Engineering

The knit scaffolds disclosed herein may be used for any purpose for which biocompatible scaffolds are desirable. For instance, the knit scaffolds may be used to repair, replace, generate, or regenerate tissue. Injuries to tissue such as cartilage, fat (i.e., adipose), skin, muscle, bone, tendon, and ligament where the tissue has been injured or traumatized frequently require surgical intervention to repair the damage and facilitate healing. Such surgical repairs can include suturing or otherwise repairing the damaged tissue with scaffolds. In some embodiments, the knit scaffolds are used to facilitate the regeneration of new, healthy tissue to provide more reliable repair and healing of the injured or damaged tissue.

In some embodiments, the current disclosure helps to address the high degree of tissue loss resulting from increasing numbers of improvised explosive devices (IEDs) encountered by members of the military. Injuries resulting in tissue loss, such as combat injuries, can be healed through tissue regeneration, which requires healthy, replication-competent cells engrafted at the site of injury. Stem cells possess high value as a tissue regenerative source by promoting proper wound healing without en masse replacement of severely injured (or missing) tissue. In addition to burn wounds, the present disclosure can be applied to more common tendon and ligament surgeries to reduce healing time. Many ligament surgeries require an autograft of tendon(s) or ligament(s) from other areas of the affected subject's body; the present work can be applied to autografted sites to promote healing and/or to injured sites where an autograft is implanted. Finally, the scaffolds disclosed herein can be applied to muscle or skin wounds for expedited healing. This may be exceptionally valuable for high-risk procedures like cardiac surgery or to expedite the repair of severe punctures and cuts.

### EXAMPLES

### Example 1. Knit Scaffolds Avoid Strain-Induced Cell Stress

FIG. 1A represents a visual schematic of the non-linear levels of stress that cells undergo over a range of strain levels, corresponding to the stretching of the structural molecules of the tissue wherein cells live. The ability of native tissue to stretch without inducing cell stress can be attributed to the naturally crimped conformation of the structural molecules (e.g., collagen fibers; see the ‘Region I’ range of the x-axis in FIG. 1A). When a moderate load is placed on the tissue, the collagen fibers begin to ‘uncrimp’, leading to the deformation of the material without stretching the collagen fibers themselves. This Region I ‘uncrimping’ is critical to the natural ability of tissue to stretch without stretching and stressing living cells locally. It is only when the collagen fibers themselves transition (see ‘Region II’ of FIG. 1A) to stretch (see ‘Region III’ of FIG. 1A) that cells begin experiencing stress on a micro level. Eventually, with enough strain, the cells die. As such, scaffold technology that allows for the movement of the fibers without straining the seeded cells, such as in the range shown in Regions I and II of FIG. 1A, is ideal.

FIG. 1B shows a comparison between currently available biocompatible scaffolds and the knit scaffolds disclosed herein. Current biocompatible traditional scaffolds that leverage hydrogels, biopolymers (e.g., collagen or fibrin), or other cell substrates capable of hosting live cells typically require a secondary skeleton for support. In addition, when exposed to strain, these traditional scaffolds stretch, causing the host cells to stretch along with them (FIG. 1B, top row). This can lead to stresses experienced by the cell, and thus a drop in cell survival, and induction of cell death. Alternatively, a knit scaffold disclosed herein has the ability to ‘uncrimp’ when exposed to strain, which avoids stretching the seeded cells (FIG. 1B, bottom row). This is achieved by knitting biocompatible threads (i.e., natural and/or polymer fibers) into a knitted conformation with the adequate micro- and macro-porosity necessary for cell seeding, cell growth, and integration with host tissue.

### Example 2. Altering Scaffold Knits for Utility in a Variety of Applications

FIG. 2 shows schematics of exemplary knit types used in the knit scaffolds described herein. Polymer fibers may be knitted in designs including, but not limited to, interlock, ribbed, or jersey knit patterns. The colored fibers in each type of knit (interlock, ribbed, and jersey) help to visualize the paths of single polymer fibers throughout the respective materials. The knit type may be further altered by varying the stitch length, which effectively modifies the number of wales, and/or number of courses in a local region of the knit. The fabric's wales direction refers to the lengthwise direction of the knit fabric, wherein the loops are formed one under the other. The fabric's courses direction refers to the crosswise direction of the knit fabric, wherein the loops are formed one after the other. The stitch length refers to the length of a single loop within the wales and courses. Specific vendors control stitch length with a stitch cam setting; in the case of the manufacturer Stoll, the stitch cam setting is set through a Needle Placement (NP) value.

### Example 3. Live Cell Growth on an Interlock-Style Knit Scaffold

FIG. 3A shows a high-magnification microscopy image of an interlock-style PLA knitted stem cell scaffold taken using a Keyence VHX-7000 digital microscope. PLA polymers were fed into an industrial knitting machine to generate an interlock-style knit scaffold. Using this scaffold, Mouse Embryonic Fibroblasts (MEFs) were seeded and allowed to grow on a knit scaffold with the same design as that shown in FIG. 3A. Before being seeded with MEFs, the knit scaffolds were washed in ethanol and/or water and then further cleaned (i.e., sprayed with 70% ethanol, then exposed to ultraviolet light for at least 2 hours). The scaffolds with cells were then cultured using Dulbecco's Modified Eagle Medium (DMEM) medium supplemented with 10% Fetal Bovine Serum (FBS) and 1% penicillin streptomycin for three days. After cells were seeded on to the scaffold, they were imaged using a Leica SP8 confocal microscope. To indicate cell viability, these cells were transfected to express a green fluorescent protein tagged nuclear localization signal which only localizes in cell nuclei when cells are alive (shown as in FIG. 3B). Additional images were taken with the confocal microscope without exposing the scaffold device to white light, in order to achieve a better overview of all live cells present in the field of view (see FIG. 3C). FIG. 3D shows the experimental setup used to collect the images in FIGS. 3B-3C. A Leica SP8 confocal microscope was outfitted with a temperature- and CO₂-controlled chamber, and used to image the scaffold device at 63× magnification. The scaffold device, including seeded cells, was cultured in a 10 cm² culture dish.

### Example 4. The Effect of Varying Knit Types on Cell Survival of Knit Scaffold-Cultured Cells

As shown in FIG. 4., mouse embryonic fibroblasts were seeded on scaffolds knitted using jersey, interlock, or ribbed knit types (at a density ˜100 cell per mm² area) and allowed to grow for 3 days in Dulbecco's Modified Eagle Medium (DMEM) medium supplemented with 10% Fetal Bovine Serum (FBS) and 1% penicillin streptomycin. All tested scaffolds had a stitch length of 13 NP as a stitch cam setting set by the Stoll knitting machine they were created on. Scaffolds were stretched by hand at nominally 1 Hz in either the wales or courses direction with a strain amplitude of 30%. After 10 minutes, the percentage of live cells compared to dead cells was measured in post-processing using ImageJ (NIH) to calculate the survival percentage compared to the initial condition before stretching. The percentage of cells that survived was comparable across all knit types at 30% strain amplitude.

### Example 5. The Effect of Varying Stitch Length on Cell Survival of Knit Scaffold-Cultured Cells

As shown in FIG. 5., mouse stem cells were seeded on interlock knit scaffolds using 9 NP, 10 NP, or 11 NP stitch lengths (For Stoll flat knitting machines, the stitch cam setting is represented by the Needle Placement (NP) value, cells seeded at a density ˜100 cell per mm² area) and allowed to grow for 3 days in Dulbecco's Modified Eagle Medium (DMEM) medium supplemented with 10% Fetal Bovine Serum (FBS) and 1% penicillin streptomycin. Scaffolds were stretched by hand at nominally 1 Hz in either the wales or courses direction with a strain amplitude of 50%. After 10 minutes, the percentage of live cells compared to dead cells was measured in post-processing using ImageJ (NIH) to calculate the survival percentage compared to the initial condition before stretching. The percentage of cells that survived was comparable across all knit types at 50% strain amplitude.

### Example 6. The Effect of Pre-Stretch and/or Repeated Stretching on Knitted Properties

FIG. 6A represents a stretching profile to investigate the effect of an initial stretching amplitude that is larger than the subsequent cyclic stretching amplitude. The profile uses an initial stretch of 60% strain of an interlock 11 NP knit with 3 ends of 64 filament/167 dtex PLA yarn, followed by repeated loading at 30% and 50%.

FIGS. 6B-6C show the results of the profile from FIG. 6A plotted as a function of stress, strain, time and stretch direction (wales vs. courses). The initial stretch demonstrates intermittent and sharp variations in response as the knit re-distributes stress in to a more uniform and balanced state. Upon subsequent loading to 30% and 50%, the cyclic loading/unloading curve is repeatable with overall lower values of stress per strain. Some residual redistribution of stress is still evident in FIG. 6C as the cyclic profile is run, but the effect is minimal compared to the initial over-stretch loading. These results demonstrate that an initial loading and/or repeated cyclic stretching can affect the final properties in an end use case. The response does, however, stabilize to a repeatable state.

### Example 7. Measurements of Stress Versus Strain of Knit Scaffolds with Varying Stitch Type

FIGS. 7A-7B demonstrate that a change in knit type between Interlock, Ribbed, and Jersey can affect the stress-strain curve of a knit—particularly in the wales direction. Knits can be changed from Ribbed, to Interlock, to Jersey to create progressively more stiff designs. Additionally, changing the knit type can adjust the anisotropy of the knit. All knits used 3 ends of 64 filament/167 dtex PLA yarn. The stitch cam settings of 10 NP for interlock, 10 NP for ribbed, and 13 NP for jersey produced stitch lengths of 5.97 mm, 6.02 mm, and 6.11 mm, respectively (stitch length defined as yarn length per stitch) to keep stitch length as nominally constant throughout testing.

### Example 8. Measurements of Stress Versus Strain of Knit Scaffolds with Varying Stitch Length

FIGS. 8A-8B demonstrates the effects of a change in stitch length as controlled by a stitch cam setting (For Stoll flat knitting machines, the stitch cam setting is represented by the Needle Placement, NP, value) on mechanical response of PLA knits. Increased stitch length lowers the stiffness of knits, primarily in the courses direction. By adjusting one direction's stiffness more heavily than the other, a change in stitch length can also adjust anisotropy. All knits used 3 ends of 64 filament/167 dtex PLA yarn. The variations of stitch length from 9 NP, 10 NP, and 11 NP produced average stitch lengths of 4.83 mm, 5.59 mm and 6.35 mm, respectively.

### Example 9. Measurements of Stress Versus Strain of Knit Scaffolds with Varying Numbers of Ends Plied Together (i.e. Variations in Numbers of Filaments)

FIGS. 9A-9B demonstrates the effects of a change in ends plied together on stress-strain curves of PLA knits. One end is 64 filament/167 dtex PLA yarn and knit type was a constant 10 NP Interlock knit. More ends produce higher ultimate stresses. To create these results, the cross-sectional area of the knit was measured as the outside boundaries of the fabric, rather than measuring the cross-sectional area of every individual filament.

### Example 10. Measurements of Stress Versus Strain of Knit Scaffolds with Varying Stitch Type

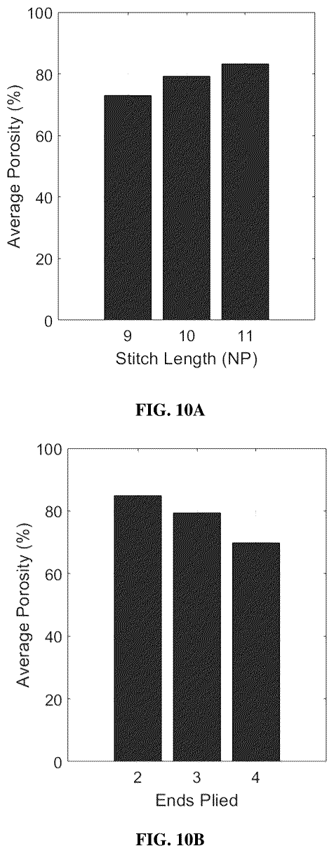

FIGS. 10A-10B demonstrates the effects of a change in stitch length and ends plied together on porosity. Porosity is defined as the ratio (presented as a percentage) of the porous media divided by the same volume of a full uniform material. Thickness of the materials were measured per ASTM D1777. The variations of stitch length from 9 NP, 10 NP, and 11 NP produced average stitch lengths of 4.83 mm, 5.59 mm and 6.35 mm, respectively. One end is 64 filament/167 dtex PLA yarn and knit type was a constant 10 NP Interlock knit. Increased stitch length increases porosity while more filaments in the base yarn decreases porosity.

### EMBODIMENTS

Embodiment 1. A three-dimensional knit scaffold comprising biocompatible fibers arranged in a knit sequence and comprising a microscopic and macroscopic porosity, wherein the scaffold has elasticity and/or anisotropy values matched to a target tissue.

Embodiment 2. The scaffold of embodiment 1, wherein the scaffold has three characteristic regions of a stress/strain curve which mimic natural tissue and correspond to ‘uncrimping’ of the knit (Region I, FIG. 1A), transitioning to stretch (Region II, FIG. 1A), and stretching of the fibers which compose the knit (Region III, FIG. 1A).

Embodiment 3. The scaffold of embodiment 1, wherein the target tissue under recovery is skin and a matched knit scaffold elasticity value of Region III (in reference to FIG. 1A) is 5 Pa to 10 MPa, and a skin anisotropy value is between 1:1.01 to 1:10.

Embodiment 4. The scaffold of embodiment 1, wherein the target tissue under recovery is ligament and a matched knit scaffold elasticity value of Region III (in reference to FIG. 1A) is 1 Mpa to 10 Gpa, and a ligament anisotropy value is between 1:1.01 to 1:10.

Embodiment 5. The scaffold of embodiment 1, wherein the target tissue under recovery is muscle and a matched knit scaffold elasticity value of Region III (in reference to FIG. 1A) is 1 kPa to 10 Mpa, and a muscle anisotropy value is between 1:1.01 to 1:10.

Embodiment 6. The scaffold of embodiment 1, wherein the target tissue under recovery is cartilage and a matched knit scaffold elasticity value of Region III (in reference to FIG. 1A) is 1 kPa to 10 Mpa, and a cartilage anisotropy value is between 1:1.01 to 1:10.

Embodiment 7. The scaffold of embodiment 1, wherein the target tissue under recovery is adipose and a matched knit scaffold elasticity value of Region III (in reference to FIG. 1A) is 1 Pa to 100 kPa, and an adipose anisotropy value is between 1:1.01 to 1:10.

Embodiment 8. The scaffold of embodiment 1, wherein the target tissue under recovery is bone and a matched knit scaffold elasticity value of Region III (in reference to FIG. 1A) is 10 Mpa to 200 Gpa, and a bone anisotropy value is between 1:1.01 to 1:10.

Embodiment 9. The scaffold of any one of embodiments 1-8, wherein the three-dimensional knit scaffold further comprises cells growing on the scaffold itself.

Embodiment 10. The scaffold of any one of embodiments 1-9, wherein a knit structure, knit type, stitch length, knit stitches per length, number of filaments in a single ‘end’ of thread, number of ends plied together in a single yarn, filament denier, overall yarn size, and/or material vary to match the biomechanical properties of the target tissue.

Embodiment 11. The scaffold of embodiment 10, wherein the knit type is selected from the group consisting of jersey knit, ribbed knit, and interlock knit.

Embodiment 12. The scaffold of any one of embodiments 1-11, wherein the biocompatible fiber is selected from the group comprising polylactic acid (PLA), polyglycolic acid (PGA), polylactic-co-glycolic acid (PLGA), poly-l-lactic acid (PLLA), poly-d-lactic acid (PLDA), Polydioxanone (PDS or PDO), Polycaprolactone (PCL), Trimethylene carbonate (TMC), and blends thereof.

Embodiment 13. The scaffold of embodiments 1-12, wherein the scaffold is comprised of a combination of PLLA, PLDA, and/or PLGA with L-Lactide (PLLA) and D-lactide (PLDA) crystallinities both ranging from 0% to 100% crystallinity.

Embodiment 14. The scaffold of any one of embodiments 1-13, wherein the scaffold has a stitch length of between 1 mm to 25 mm.

Embodiment 15. The scaffold of any one of embodiments 1-14, wherein the scaffold has a number of filaments combined into a base ‘end’ of fiber ranging from between 1-500 filaments.

Embodiment 16. The scaffold of any one of embodiments 1-15, wherein the scaffold has a number of fiber ends plied together in a single yarn having a value of between 1-10.

Embodiment 17. The scaffold of any one of embodiments 1-16, wherein the scaffold has a filament denier of between 1-400.

Embodiment 18. The scaffold of any one of embodiments 1-17, wherein the scaffold has a degradation rate of between 1 week to 5 years.

Embodiment 19. A method for preparing a tissue scaffold, comprising:
Identifying a set of values defining an elasticity (defined in Regions I, II, and/or III from FIG. 1A), anisotropy, healing rate, and/or hydraulic permeability value of a target tissue, and
knitting mono-filament or multi-filament biocompatible fibers to produce a scaffold arranged in a knit sequence and comprising a microscopic porosity sufficient to nurture growing cells compatible with target tissue recovery, macroscopic porosity matched to the required hydraulic permeability of the target tissue, degradation rate which degrades at a similar rate to the healing rate of the target tissue, and having an elasticity (as defined by Regions I, II, and/or III in FIG. 1A) and anisotropy biomechanical values matched to that of the target tissue. The knits may be optionally not stretched, pre-over-stretched, or cyclically stretched to achieve final elasticity values of host tissue (i.e., to prime the scaffold for use). Additionally, the knits may be optionally heat set to different times and temperatures to achieve final elasticity values of host tissue (i.e., to prime the scaffold for use).

Embodiment 20. The method of embodiment 19, further comprising seeding the scaffold with human-derived tissue, blood, bone marrow, platelet rich plasma, platelets, mononuclear cells, progenitor cells, inflammatory cells, primary cells, stem cells, induced pluripotent stem cells or purified cell populations, fluids, or proteins.

Embodiment 21. The method of embodiment 19 or 20, further comprising seeding the scaffold with cells selected from the group consisting of chondrocytes, osteoblasts, fibroblasts, angioblasts, myoblasts, epithelial cells, urothelial cells, smooth muscle cells, keratinocytes, beta cells, endothelial cells, fibrocytes, vascular endothelial cells, hepatocytes, small intestine epithelial cells, epidermal keratinocytes, bone marrow mesenchymal cells, cardiomyocytes, intervertebral disc cells, oral mucosal epithelia, gastrointestinal mucosal epithelia, urinary tract epithelia, skeletal joint synovium, periosteum, perichondrium, skeletal muscle cells, smooth muscle cells, cardiac muscle cells, pericardium, dura, meninges, keratinocyte precursor cells, pericytes, glial cells, neural cells, amniotic membrane, placental membrane, serosal cells, undifferentiated stem cells, undifferentiated progenitor cells, pre-differentiated stem cells, pre-differentiated progenitor cells, neural stem cells, neural progenitor cells, neuronal cells, dendritic cells, genetically engineered cells, and/or stem cells.

Embodiment 22. The method of embodiment 21, wherein the stem cells are selected from the group consisting of hematopoietic, mesenchymal, postpartum, pancreatic, hepatic, retinal epithelial, olfactory bulb, endothelial, muscle, adipose derived, ileac crest, bone marrow, periodontal ligament, oval and dermal cells and organ specific stem and/or progenitor cells.

Embodiment 23. The method of any one of embodiments 19-22, wherein a knit type, stitch length, number of stitches per length, number of filaments combined in to a base ‘end’ of fiber, number of fiber ends plied together in a single yarn, filament denier, overall yarn size, and/or material is determined based upon the properties of the target tissue and used to produce the scaffold.

Embodiment 24. A method of treating a subject in need of tissue generation comprising
contacting the subject with a scaffold of any one of embodiments 1-18, and allowing tissue to generate on the scaffold.

Embodiment 25. The method of embodiment 24, wherein the subject is a human.

Embodiment 26. The method of embodiment 24 or 25, wherein the tissue is skin, muscle, ligament, cartilage, adipose, and/or bone.

Embodiment 27. The scaffold of any one of embodiments 1-8, wherein the three-dimensional knit scaffold further comprises Collagen Type I, II, III, IV, and/or V on the surface of the knit.

Embodiment 28. The scaffold of any one of embodiments 1-8, wherein the three-dimensional knit scaffold does not comprise Collagen Type I, II, III, IV, and/or V on the surface of the knit.

Embodiment 29. The method of embodiment 24, wherein Collagen Type I, II, III, IV, and/or V is added to the surface of the knit prior to seeding with cells.

1. A three-dimensional knit scaffold comprising biocompatible fibers arranged in a knit sequence and comprising a microscopic and macroscopic porosity, wherein the scaffold has an elasticity value and an anisotropy value matched to a target tissue.

2. The three-dimensional knit scaffold of claim 1, wherein the three-dimensional knit scaffold has three characteristic regions of a stress/strain curve which mimic the target tissue and correspond to:
(i) uncrimping of the knit;
(ii) transitioning to stretch; and
(iii) stretching of the fibers which compose the knit.

3. The three-dimensional knit scaffold of claim 1, wherein:
the target tissue is skin, and the elasticity value is between 5 Pa to 10 MPa and the anisotropy value is between 1:1.01 to 1:10;
the target tissue is ligament, and the elasticity value is between 1 MPa to 10 GPa and the anisotropy value is between 1:1.01 to 1:10;
the target tissue is muscle, and the elasticity value is between 1 kPa to 10 MPa and the anisotropy value is between 1:1.01 to 1:10;
the target tissue is cartilage, and the elasticity value is between 1 kPa to 10 MPa and the anisotropy value is between 1:1.01 to 1:10;
the target tissue is adipose, and the adipose-matched scaffold elasticity value is 1 Pa to 100 kPa and the anisotropy value is 1:1.01 to 1:10; or
the target tissue is bone, and the elasticity value is between 10 MPa to 200 GPa and the anisotropy value is between 1:1.01 to 1:10.

4-8. (canceled)

9. The three-dimensional knit scaffold of claim 1, wherein the three-dimensional knit scaffold further comprises cells growing on the scaffold.

10. The three-dimensional knit scaffold of claim 1, wherein a collagen is deposited on the three-dimensional knit scaffold before cells are seeded on the three-dimensional knit scaffold, optionally wherein the collagen is Collagen Type I, II, III, IV, and/or V.

11. (canceled)

12. The three-dimensional knit scaffold of claim 1, wherein a knit structure, knit type, stitch length, number of stitches per length, number of filaments in a single ‘end’ of thread, number of ends plied together in a single yarn, filament denier, overall yarn size, and/or material are varied to match biomechanical properties of the target tissue, optionally wherein the knit type is selected from a jersey knit, ribbed knit, or interlock knit.

13. (canceled)

14. The three-dimensional knit scaffold of claim 1, wherein the biocompatible fiber:
is selected from the group comprising polylactic acid (PLA), polyglycolic acid (PGA), polylactic-co-glycolic acid (PLGA), poly-l-lactic acid (PLLA), poly-d-lactic acid (PLDA), Polydioxanone (PDS or PDO), Polycaprolactone (PCL), Trimethylene carbonate (TMC), and blends thereof; and/or
comprises PLLA, PLDA, and/or PLGA, optionally wherein a crystallinity of PLLA and/or PLDA are between 0% to 100%.

15-16. (canceled)

17. The three-dimensional knit scaffold of claim 1, wherein:
the three-dimensional knit scaffold has a stitch length of between 1 mm to 25 mm;
between 1-500 filaments are combined into a base end of the biocompatible fiber;
1-10 fiber ends are plied together in a single yarn;
the three-dimensional knit scaffold comprises a filament denier of between 5-400; and/or
the three-dimensional knit scaffold has a degradation rate of between 1 week to 5 years.

18-21. (canceled)

22. The three-dimensional knit scaffold of claim 1, comprising a shape and/or a pattern that matches an area of the body where the scaffold is implemented.

23. A method for preparing a tissue scaffold, comprising:
identifying a set of values defining an elasticity, anisotropy, healing rate, and/or hydraulic permeability value of a target tissue, and
knitting monofilament or multifilament biocompatible fibers to produce a scaffold arranged in a knit sequence and comprising a microscopic porosity sufficient to support growing cells compatible with target tissue recovery, macroscopic porosity matched to the required hydraulic permeability of the target tissue, degradation rate which degrades at a similar rate to the healing rate of the target tissue, and having anisotropy biomechanical values matched to that of the target tissue.

24. The method of claim 23, further comprising a step of priming the tissue scaffold for use, optionally wherein the priming comprises:
over-stretching the tissue scaffold;
cyclically stretching the tissue scaffold; and/or
heating the tissue scaffold.

25-26. (canceled)

27. The method of claim 23, further comprising seeding the tissue scaffold with human-derived tissue, blood, bone marrow, platelet rich plasma, platelets, mononuclear cells, progenitor cells, inflammatory cells, primary cells, stem cells, induced pluripotent stem cells or purified cell populations, fluids, and/or proteins.

28. The method of claim 23, wherein a collagen is deposited on the tissue scaffold before cells are seeded on the scaffold, optionally wherein the collagen is Collagen Type I, II, III, IV, and/or V.

29. (canceled)

30. The method of claim 23, further comprising seeding the tissue scaffold with cells selected from the group comprising chondrocytes, osteoblasts, fibroblasts, angioblasts, myoblasts, epithelial cells, urothelial cells, smooth muscle cells, keratinocytes, beta cells, endothelial cells, fibrocytes, vascular endothelial cells, hepatocytes, small intestine epithelial cells, epidermal keratinocytes, bone marrow mesenchymal cells, cardiomyocytes, intervertebral disc cells, oral mucosal epithelia, gastrointestinal mucosal epithelia, urinary tract epithelia, skeletal joint synovium, periosteum, perichondrium, skeletal muscle cells, smooth muscle cells, cardiac muscle cells, pericardium, dura, meninges, keratinocyte precursor cells, pericytes, glial cells, neural cells, amniotic membrane, placental membrane, serosal cells, undifferentiated stem cells, undifferentiated progenitor cells, pre-differentiated stem cells, pre-differentiated progenitor cells, neural stem cells, neural progenitor cells, neuronal cells, dendritic cells, genetically engineered cells, and stem cells, optionally wherein the stem cells are selected from the group comprising hematopoietic, mesenchymal, postpartum, pancreatic, hepatic, retinal epithelial, olfactory bulb, endothelial, muscle, adipose derived, ileac crest, bone marrow, periodontal ligament, oval and dermal cells and organ specific stem and progenitor cells.

31. (canceled)

32. The method of claim 23, wherein:
the target tissue is skin, the elasticity value is between 5 Pa to 10 MPa and the anisotropy value is between 1:1.01 to 1:10;
the target tissue is ligament, and the elasticity value is between 1 MPa to 10 GPa and the anisotropy value is between 1:1.01 and 1:10;
the target tissue is muscle, and the elasticity value is between 1 kPa and 10 MPa and the anisotropy value is between 1:1.01 and 1:10;
the target tissue is cartilage, and the elasticity value is between 1 kPa to 10 MPa and the anisotropy value is between 1:1.01 to 1:10;
the target tissue is adipose, and the elasticity value is between 1 Pa to 100 kPa and the anisotropy value is between 1:1.01 to 1:10; or
the target tissue is bone, and the elasticity value is between 10 MPa to 200 GPa and the anisotropy value is between 1:1.01 to 1:10.

33-37. (canceled)

38. The method of claim 23, wherein a knit type, a stitch length, a number of stitches per length, a number of filaments combined in a base ‘end’ of fiber, a number of fiber ends plied together in a single yarn, a filament denier, an overall yarn size, and/or a material is determined based upon properties of the target tissue and used to produce the scaffold.

39. A method of treating a subject in need of tissue generation comprising
contacting the subject with the three-dimensional knit scaffold of claim 1 and allowing tissue to generate on the three-dimensional knit scaffold.

40. The method of claim 39 wherein the subject is a human.

41. The method of claim 39, wherein the tissue is skin, muscle, ligament, cartilage, adipose, and/or bone.

42. The three-dimensional knit scaffold of claim 1, wherein the three-dimensional knit scaffold is primed for use, optionally wherein the priming comprises:
over-stretching the three-dimensional knit scaffold;
cyclically stretching the three-dimensional knit scaffold; and/or
heating the three-dimensional knit scaffold.
